(19) United States
(12) Patent Application Publication
(10) Pub. No.: US 2004/0023336 A1
(43) Pub. Date: Feb. 5, 2004

(54) Mut-IL-18 or Mut-IL-18R proteins, antibodies, compositions, methods and uses

(76) Inventors: George A. Heavner, Malvern, PA (US); Linda A. Snyder, Pottstown, PA (US); Stephen G. McCarthy, West Chester, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/280,609

(22) Filed: Oct. 25, 2002

Related U.S. Application Data
(60) Provisional application No. 60/335,880, filed on Oct. 26, 2001.

(51) Int. Cl. C07H 21/04; C12P 21/04; C12P 21/02; C12N 5/06; C07K 14/54

(52) U.S. Cl. 435/69.52; 435/320.1; 435/325; 530/351; 536/23.5

(57) ABSTRACT

The present invention relates to at least one novel Mut-IL18 or Mut-IL-18R proteins, antibodies, including isolated nucleic acids that encode at least one Mut-IL18 or Mut-IL-18R protein or antibody, Mut-IL18 or Mut-IL-18R vectors, host cells, transgenic animals or plants, and methods of making and using thereof, including therapeutic compositions, methods and devices.

### BACKGROUND OF THE INVENTION

1. Field of the Invention

This application is based in part on, and claims priority to, U.S. Provisional No. 60/335,880 filed Oct. 26, 2001, which is entirely incorporated herein by reference.

The present invention relates to at least one Interleukin-18/Interleukin receptor mutein (Mut-IL18 or Mut-IL-18R) protein or fragment thereof, and antibodies, including specified portions or variants, specific therefore, as well as nucleic acids encoding such Mut-IL18 or Mut-IL-18R proteins, fragments, antibodies, complementary nucleic acids, vectors, host cells, and methods of making and using thereof, including therapeutic formulations, administration and devices.

2. Related Art

IL-18 is a proinflammatory cytokine that is able to induce IFNγ, GM-CSF, TNF?, and IL-1 in immunocompetent cells. IL-18 can activate killing by lymphocytes, and can upregulate the expression of certain chemokine receptors. IL-18 is essential to host defense against severe infections, because it can induce IFNγ that in turn evokes effector molecules such as nitric oxide. IL-18 induces cytotoxic T cells, which can be specifically directed against viral antigens. IL-18 also enhances tumor rejection by its ability to augment the cytotoxic activity of NK and T cells in vivo. IL-18 responsiveness of a human myelomonocytic cell line KG-1 cells has recently been reported. It has also recently been demonstrated that IL-18 induces IFN?, ICAM-1 and CD95 expression on primary murine macrophages. Both of these findings indicate that monocytes express and signal via IL-18r. In order to understand further the importance of IL-18 stimulation of the macrophage compartment, we conducted a gene expression microarray of KG-1 cells stimulated by IL-18. The most striking result was that several mature dendritic cell (DC) related genes were up regulated by IL-18. It is now accepted that monocytes differentiate into dendritic ells under certain conditions. In vitro this is achieved by GMCSF+IL-4 and further matured with LPS, TNF, IL-1 and PGE2. DCs are antigen-presenting cells that specialize in the initiation of T-cell response in vivo by presenting peptide in the context of MHC along with appropriate costimulation. The finding that IL-18 is directly involved in the induction and maturation of dendritic cells is both novel and unexpected and indicates this cytokine is a key regulator of immune responses involved in increasing antigen presentation via direct induction of DC activity.

Clearly manipulation of IL-18 activity can affect the course of various immune imbalances. The ability to design muteins of IL-18 with either agonistic or antagonistic activity and to raise antibodies against IL-18 is important in the discovery and development of agents to alter IL-18 activity.

Such Mut-IL18 or Mut-IL-18R proteins can potentially be further engineered to provide enhanced properties, such as increased or modified biological half lives, modified biological activities, enhanced immungenicity for generating antibodies, increased stability or expression, and the like.

Non-human mammalian, chimeric, polyclonal (e.g., sera) and/or monoclonal antibodies (Mabs) and fragments (e.g., proteolytic digestion or fusion protein products thereof) are potential therapeutic agents that are being investigated in some cases to attempt to treat certain diseases. However, such antibodies or fragments can elicit an immune response when administered to humans. Such an immune response can result in an immune complex-mediated clearance of the antibodies or fragments from the circulation, and make repeated administration unsuitable for therapy, thereby reducing the therapeutic benefit to the patient and limiting the readministration of the antibody or fragment. For example, repeated administration of antibodies or fragments comprising non-human portions can lead to serum sickness and/or anaphalaxis. In order to avoid these and other problems, a number of approaches have been taken to reduce the immunogenicity of such antibodies and portions thereof, including chimerization and humanization, as well known in the art. These and other approaches, however, still can result in antibodies or fragments having some immunogenicity, low affinity, low avidity, or with problems in cell culture, scale up, production, and/or low yields. Thus, such antibodies or fragments can be less than ideally suited for manufacture or use as therapeutic proteins.

Accordingly, there is a need to provide Mut-IL18 or Mut-IL-18R proteins or antibodies or fragments that overcome one more of these problems, as well as improvements over known proteins or antibodies or fragments thereof.

### SUMMARY OF THE INVENTION

The present invention provides isolated human, primate, rodent, mammalian, chimeric, or human Mut-IL18 or Mut-IL-18R proteins, antibodies, immunoglobulins, cleavage products and other specified portions and variants thereof, as well as Mut-IL18 or Mut-IL-18R protein or anibody compositions, encoding or complementary nucleic acids, vectors, host cells, compositions, formulations, devices, transgenic animals, transgenic plants, and methods of making and using thereof, as described and enabled herein, in combination with what is known in the art.

The present invention also provides at least one isolated Mut-IL18 or Mut-IL-18R antibody as described herein. An antibody according to the present invention can include any protein or peptide containing molecule that comprises at least a portion of an immunoglobulin molecule, such as but not limited to at least one complementarity determinng region (CDR) (also termed the hypervariable region or HV) of a heavy or light chain variable region, or a ligand binding portion thereof, a heavy chain or light chain variable region, a heavy chain or light chain constant region, a framework region, or any portion thereof, wherein the antibody can be incorporated into an antibody of the present invention. An antibody of the invention can include or be derived from any mammal, such as but not limited to a human, a mouse, a rabbit, a rat, a rodent, a primate, or any combination thereof, and the like.

The present invention provides, in one aspect, isolated nucleic acid molecules comprising, complementary, or hybridizing to, a polynucleotide encoding specific Mut-IL18 or Mut-IL-18R proteins or antibodies, comprising at least one specified sequence, domain, portion or variant thereof. The present invention further provides recombinant vectors comprising at least ibe if said Mut-IL18 or Mut-IL-18R protein or antibody encoding or complementary nucleic acid molecules, host cells containing such nucleic acids and/or recombinant vectors, as well as methods of making and/or using such antibody nucleic acids, vectors and/or host cells.

At least one antibody of the invention binds at least one specified epitope specific to at least one Mut-IL18 or Mut-IL-18R protein, subunit, fragment, portion or any combination thereof. The at least one epitope can comprise at least one antibody binding region that comprises at least one portion of said protein, which epitope is preferably comprised of at least 1-5 amino acids of at least one portion thereof, such as but not limited to, at least one functional, extracellular, soluble, hydrophillic, external or cytoplasmic domain of said protein, or any portion thereof.

The at least one antibody can optionally comprise at least one specified portion of at least one complementarity determining region (CDR) (e.g., CDR1, CDR2 or CDR3 of the heavy or light chain variable region) and optionally at least one constant or variable framework region or any portion thereof. The at least one antibody amino acid sequence can further optionally comprise at least one specified substitution, insertion or deletion as described herein or as known in the art.

The present invention also provides at least one isolated Mut-IL18 or Mut-IL-18R protein or antibody as described herein, wherein the antibody has at least one activity, such as, but not limited to known IL-18 or IL-18R activities. A(n) Mut-IL18 or Mut-IL-18R protein antibody can thus be screened for a corresponding activity according to known methods, such as but not limited to, at least one biological activity towards a Mut-IL18 or Mut-IL-18R protein or protein related function.

The present invention further provides at least one Mut-IL18 or Mut-IL-18R anti-idiotype antibody to at least one Mut-IL18 or Mut-IL-18R antibody of the present invention. The anti-idiotype antibody includes any protein or peptide containing molecule that comprises at least a portion of an immunoglobulin molecule, such as but not limited to at least one complementarity determinng region (CDR) of a heavy or light chain or a ligand binding portion thereof, a heavy chain or light chain variable region, a heavy chain or light chain constant region, a framework region, or any portion thereof, that can be incorporated into an antibody of the present invention. An antibody of the invention can include or be derived from any mammal, such as but not limited to a human, a mouse, a rabbit, a rat, a rodent, a primate, and the like. The present invention provides, in one aspect, isolated nucleic acid molecules comprising, complementary, or hybridizing to, a polynucleotide encoding at least one Mut-IL18 or Mut-IL-18R anti-idiotype antibody, comprising at least one specified sequence, domain, portion or variant thereof. The present invention further provides recombinant vectors comprising said Mut-IL18 or Mut-IL-18R anti-idiotype antibody encoding nucleic acid molecules, host cells containing such nucleic acids and/or recombinant vectors, as well as methods of making and/or using such anti-idiotype antiobody nucleic acids, vectors and/or host cells.

The present invention also provides at least one method for expressing at least one Mut-IL18 or Mut-IL-18R protein or antibody, or Mut-IL18 or Mut-IL-18R anti-idiotype antibody, in a host cell, comprising culturing a host cell as described herein under conditions wherein at least one Mut-IL18 or Mut-IL-18R antibody is expressed in detectable and/or recoverable amounts.

The present invention also provides at least one composition comprising (a) an isolated Mut-IL18 or Mut-IL-18R protein or antibody encoding nucleic acid and/or protein or antibody as described herein; and (b) a suitable carrier or diluent. The carrier or diluent can optionally be pharmaceutically acceptable, such as but not limited to known carriers or diluents. The composition can optionally further comprise at least one further compound, protein or composition.

The present invention further provides at least one Mut-IL18 or Mut-IL-18R protein or antibody method or composition, for administering a therapeutically effective amount to modulate or treat at least one Mut-IL18 or Mut-IL-18R related condition in a cell, tissue, organ, animal or patient and/or, prior to, subsequent to, or during a related condition, as known in the art and/or as described herein.

The present invention also provides at least one composition, device and/or method of delivery of a therapeutically or prophylactically effective amount of at least one Mut-IL18 or Mut-IL-18R protein or antibody, according to the present invention.

The present invention further provides at least one Mut-IL18 or Mut-IL-18R protein or antibody method or composition, for diagnosing at least one Mut-IL18 or Mut-IL-18R related condition in a cell, tissue, organ, animal or patient and/or, prior to, subsequent to, or during a related condition, as known in the art and/or as described herein.

The present invention also provides at least one composition, device and/or method of delivery for diagnosing of at least one Mut-IL18 or Mut-IL-18R protein or antibody, according to the present invention.

In another aspect, the present invention provides at least one isolated mammalian Mut-IL18 or Mut-IL-18R protein, comprising at least one variable region comprising at least one of SEQ ID NOS: 1-2.

In another aspect, the present invention provides at least one isolated mammalian Mut-IL18 or Mut-IL-18R protein, comprising the amino acid sequences as part of at least one of SEQ ID NOS: 1-2.

Also provided is an isolated nucleic acid encoding at least one isolated mammalian Mut-IL18 or Mut-IL-18R protein; an isolated nucleic acid vector comprising the isolated nucleic acid, and/or a prokaryotic or cukaryotic host cell comprising the isolated nucleic acid. The host cell can optionally be at least one selected from prokaryotic or eukaryotic cells, or fusion cells thereof, e.g., but not limited to, mammalian, plant or insect, such as but not limited to, CHO, myeloma, or lymphoma cells, bacterial cells, yeast cells, silk worm cells, or any derivative, immortalized or transformed cell thereof. Also provided is a method for producing at least one Mut-IL18 or Mut-IL-18R protein, comprising translating the protein encoding nucleic acid under conditions in vitro, in vivo or in situ, such that the Mut-IL18 or Mut-IL-18R protein is expressed in detectable or recoverable amounts.

Also provided is a composition comprising at least one isolated mammalian Mut-IL18 or Mut-IL-18R protein and at least one pharmaceutically acceptable carrier or diluent. The composition can optionally further comprise an effective amount of at least one compound or protein selected from at least one of a detectable label or reporter, a TNF antagonist, an antirheumatic, a muscle relaxant, a narcotic, a non-steroid inflammatory drug (NTHE), an analgesic, an anesthetic, a sedative, a local anethetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

Also provided is a method for diagnosing or treating a Mut-IL18 or Mut-IL-18R related condition in a cell, tissue, organ or animal, comprising

(a) contacting or administering a composition comprising an effective amount of at least one isolated mammalian Mut-IL18 or Mut-IL-18R protein of the invention with, or to, the cell, tissue, organ or animal. The method can optionally further comprise using an effective amount of 0.0000001-500 mg/kilogram of the cells, tissue, organ or animal. The method can optionally further comprise using the contacting or the administrating by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal. The method can optionally further comprise administering, prior, concurrently or after the (a) contacting or administering, at least one composition comprising an effective amount of at least one compound or protein selected from at least one of a detectable label or reporter, a TNF antagonist, an antirheumatic, a muscle relaxant, a narcotic, an anti-inflammatory, a non-steroid inflammatory drug (NTHE), an analgesic, an anesthetic, a sedative, a local anethetic, a neuromuscular blocker, an antimicrobial, an antipsoriatic, a corticosteriod, an anabolic steroid, an erythropoietin, an immunization, an immunoglobulin, an immunosuppressive, a hormone, a hormone replacement drug, a radiopharmaccutical, an antidepressant, an antipsychotic, a stimulant, an asthma medication, a beta agonist, an inhaled steroid, an epinephrine or analog, a cytokine, or a cytokine antagonist.

Also provided is at least one medical device, comprising at least one isolated mammalian Mut-IL18 or Mut-IL-18R protein of the invention, wherein the device is suitable to contacting or administerting the at least one Mut-IL18 or Mut-IL-18R protein by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal.

Also provided is an article of manufacture for human pharmaceutical or diagnostic use, comprising packaging material and a container comprising a solution or a lyophilized form of at least one isolated mammalian Mut-IL18 or Mut-IL-18R protein of the present invention. The article of manufacture can optionally comprise having the container as a component of a parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal delivery device or system.

Also provided is a method for producing at least one isolated mammalian Mut-IL18 or Mut-IL-18R protein of the present invention, comprising providing a host cell or transgenic animal or transgenic plant or plant cell capable of expressing in recoverable amounts the protein. Further provided in the present invention is at least one Mut-IL18 or Mut-IL-18R protein produced by the above method.

In other aspect the present invention provides at least one isolated mammalian Mut-IL-18R or Mut-IL-18R antibody, comprising at least one human CDR, wherein the antibody specifically binds at least one epitope comprising at least 1-3, to the entire amino acid sequence of SEQ ID NOS: 1-2.

The at least one antibody can optionally further at least one of: bind Mut-IL18 or Mut-IL-18R with an affinity of at least one selected from at least 10⁻⁹ M, at least 10⁻¹⁰ M, at least 10⁻¹¹ M, or at least 10⁻¹² M; substantially neutralizes at least one activity of at least one Mut-IL18 or Mut-IL-18R protein. Also provided is an isolated nucleic acid encoding at least one isolated mammalian Mut-IL18 or Mut-IL-18R antibody; an isolated nucleic acid vector comprising the isolated nucleic acid, and/or a prokaryotic or eukaryotic host cell comprising the isolated nucleic acid. The host cell can optionally be at least one selected from prokaryotic or eukaryotic cells, or fusion cells thereof, e.g., but not limited to, mammalian, plant or insect, such as but not limited to, CHO, myeloma, or lymphoma cells, bacterial cells, yeast cells, silk worm cells, or any derivative, immortalized or transformed cell thereof. Also provided is a method for producing at least one Mut-IL18 or Mut-IL-18R antibody, comprising translating the antibody encoding nucleic acid under conditions in vitro, in vivo or in situ, such that the Mut-IL18 or Mut-IL-18R antibody is expressed in detectable or recoverable amounts.

Also provided is a composition comprising at least one isolated mammalian Mut-IL18 or Mut-IL-18R antibody and at least one pharmaceutically acceptable carrier or diluent. The composition can optionally further comprise an effective amount of at least one compound or protein selected from at least one of a detectable label or reporter, a TNF antagonist, an antirheumatic, a muscle relaxant, a narcotic, a non-steroid inflammatory drug (NTHE), an analgesic, an anesthetic, a sedative, a local anethetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

The present invention further provides an anti-idiotype antibody or fragment that specifically binds at least one isolated mammalian Mut-IL18 or Mut-IL-18R antibody of the present invention.

Also provided is a method for diagnosing or treating a Mut-IL18 or Mut-IL-18R related condition in a cell, tissue, organ or animal, comprising

(a) contacting or administering a composition comprising an effective amount of at least one isolated mammalian Mut-IL18 or Mut-IL-18R antibody of the invention with, or to, the cell, tissue, organ or animal. The method can optionally further comprise using an effective amount of 0.0001-500 mg/kilogram of the cells, tissue, organ or animal. The method can optionally further comprise using the contacting or the administrating by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal. The method can optionally further comprise administering, prior, concurrently or after the (a) contacting or administering, at least one composition comprising an effective amount of at least one compound or protein selected from at least one of a detectable label or reporter, a TNF antagonist, an antirheumatic, a muscle relaxant, a narcotic, an anti-inflammatory, a non-steroid inflammatory drug (NTHE), an analgesic, an anesthetic, a sedative, a local anethetic, a neuromuscular blocker, an antimicrobial, an antipsoriatic, a corticosteriod, an anabolic steroid, an erythropoietin, an immunization, an immunoglobulin, an immunosuppressive, a hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

Also provided is at least one medical device, comprising at least one isolated mammalian Mut-IL18 or Mut-IL-18R antibody of the invention, wherein the device is suitable to contacting or administerting the at least one Mut-IL18 or Mut-IL-18R antibody by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal.

Also provided is an article of manufacture for human pharmaceutical or diagnostic use, comprising packaging material and a container comprising a solution or a lyophilized form of at least one isolated mammalian Mut-IL18 or Mut-IL-18R antibody of the present invention. The article of manufacture can optionally comprise having the container as a component of a parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal delivery device or system.

Also provided is a method for producing at least one isolated mammalian Mut-IL18 or Mut-IL-18R antibody of the present invention, comprising providing a host cell or transgenic animal or transgenic plant or plant cell capable of expressing in recoverable amounts the antibody. Further provided in the present invention is at least one Mut-IL18 or Mut-IL-18R antibody produced by the above method.

The present invention further provides any invention described herein.

### DESCRIPTION OF THE FIGURES

FIG. 1 is a human IL-18 amino acid sequence.

FIG. 2A-2B is a human IL-18 receptor amino acid sequence.

### DESCRIPTION OF THE INVENTION

The present invention provides isolated, recombinant and/or synthetic Mut-IL18 or Mut-IL-18R human, primate, rodent, mammalian, chimeric, humanized or CDR-grafted, antibodies and Mut-IL18 or Mut-IL-18R anti-idiotype antibodies thereto, as well as compositions and encoding nucleic acid molecules comprising at least one polynucleotide encoding at least one Mut-IL18 or Mut-IL-18R antibody or anti-idiotype antibody. The present invention further includes, but is not limited to, methods of making and using such nucleic acids and antibodies and anti-idiotype antibodies, including diagnostic and therapeutic compositions, methods and devices.

As used herein, an “Interleukin-18/Interleukin receptor muteins antibody,” “Mut-IL18 or Mut-IL-18R antibody,” and the like include any protein or peptide containing molecule that comprises at least a portion of an immunoglobulin molecule, such as but not limited to at least one complementarity determinng region (CDR) of a heavy or light chain or a ligand binding portion thereof, a heavy chain or light chain variable region, a heavy chain or light chain constant region, a framework region, or any portion , fragment or variant thereof, or at least one portion of an Mut-IL18 or Mut-IL-18R receptor or binding protein, which can be incorporated into a Mut-IL18 or Mut-IL-18R antibody of the present invention.

Antibodies can include one or more of at least one CDR, at least one variable region, at least one constant region, at least one heavy chain (e.g., γ₁, γ₂, γ₃, γ₄, μ, α₁, α₂, δ, ε, at least one light chain (e.g., κ and λ), or any portion or fragment thereof, and can further comprise interchain and intrachain disulfide bonds, hinge regions, glycosylation sites that can be separated by a hinge region, as well as heavy chains and light chains. Light chains typically have a molecular weight of about 25Kd and heavy chains typically range from 50K-77Kd. Light chains can exist in two distinct forms or isotypes, kappa (κ) and lambda (λ), which can combine with any of the heavy chain types. All light chains have at least one variable region and at least one constant region. The IgG antibody is considered a typical antibody structure and has two intrachain disulfide bonds in the light chain (one in variable region and one in the constant region), with four in the heavy chain, and such bond encompassing a peptide loop of about 60-70 amino acids comprising a “domain” of about 110 amino acids in the chain. IgG antibodies can be characterized into four classes, IgG1, IgG2, IgG3 and IgG4. Each immunoglobulin class has a different set of functions. The following table summarizes the Physicochemical properties of each of the immunoglobuling classes and subclasses.

| Property | IgG1 | IgG2 | IgG3 | IgG4 | IgM | IgA1 | IgA2 | SIgA | IgD | IgE |
|---|---|---|---|---|---|---|---|---|---|---|
| Heavy Chain | γ1 | γ1 | γ1 | γ1 | μ | α1 | α2 | α1/α2 | δ | e |
| Mean Serum conc. | 9 | 3 | 1 | 0.5 | 1.5 | 3.0 | 0.5 | 0.05 | 0.03 | 0.00005 |
| (mg/ml) | | | | | | | | | | |
| Sedimentation | 7s | 7s | 7s | 7s | 19s | 7s | 7s | 11s | 7s | 8s |
| constant | | | | | | | | | | |
| Mol. Wt. (×10³) | 146 | 146 | 170 | 146 | 970 | 160 | 160 | 385 | 184 | 188 |
| Half Life (days) | 21 | 20 | 7 | 21 | 10 | 6 | 6 | ? | 3 | 2 |
| % intravascular | 45 | 45 | 45 | 45 | 80 | 42 | 42 | Trace | 75 | 50 |
| distribution | | | | | | | | | | |
| Carbohydrate (%) | 2-3 | 2-3 | 2-3 | 2-3 | 12 | 7-11 | 7-11 | 7-11 | 9-14 | 12 |

The following table summariizes non-limiting examples of antibody effector functions for human antibody classes and subclasses.

| Effector function | IgG1 | IgG2 | IgG3 | IgG4 | IgM | IgA | IgD | IgE |
|---|---|---|---|---|---|---|---|---|
| Complement fixation | ++ | + | +++ | − | +++ | − | − | — |
| Placental transfer | + | + | + | + | — | — | — | — |
| Binding to Staph A | +++ | +++ | — | +++ | — | — | — | — |
| Binding to Strep G | +++ | +++ | +++ | +++ | — | — | — | — |

Accordingly, the type of antibody or fragment thereof can be selected for use according to the present invention based on the desired characteristics and functions that are desired for a particular therapeutic or diagnostic use, such as but not limited to serum half life, intravascular distribution, complement fixation, etc.

Antibody diversity is generated by at leat 5 mechanisms, including (1) the use of multiple genes encoding parts of the antibody; (2) somoatic mutation, e.g., primordial V gene mutation during B-cell ontogeny to produce different V genes in different B-cell clones; (3) somatic recombination, e.g., gene segments J1-Jn recombine to join the man part of the V-region gene during B-cell ontogeny; (4) gene conversion where sections of DNA from a number of pseudo V region can be copied into the V region to alter the DNA sequence; and (5) nucleotide addition, e.g., when V and J regions are cut, before joining, and extra nucleotides may be inserted to code for additional amino acids. Non-limiting examples include, but are not limited to, (i) the selection/recombination of Vκ, J, and Cκ regions from germ line to B-cell clones to generate kappa chains; (ii) selection/recombination of Vλ, J, and Cλ regions from germ line to B-cell clones to generate lambda chains; (iii) selection/recombination of V_{H}, D1-D30 and J_{H}1-J_{H}6 genes to form a functional VDJ gene encoding a heavy chain variable region. The above mechanisms work in a coordinated fashion to generate antibody diversity and specificity.

The term “antibody” is further intended to encompass antibodies, digestion fragments, specified portions and variants thereof, including antibody mimetics or comprising portions of antibodies that mimic the structure and/or function of an anitbody or specified fragment or portion thereof, including single chain antibodies and fragments thereof. Functional fragments include antigen-binding fragments that bind to a mammalian Mut-IL18 or Mut-IL-18R. For example, antibody fragments capable of binding to Mut-IL18 or Mut-IL-18R or portions thereof, including, but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the CH₁ domain and/or hinge region of the heavy chain. The various portions of antibodies can be joined together chemically by conventional techniques, or can be prepared as a contiguous protein using genetic engineering techniques.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. Similarly, antibodies designated primate (monkey, babboon, chimpanzee, etc.), rodent (mouse, rat, rabbit, guinea pid, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Bispecific, heterospecific, heteroconjugate or similar antibodies can also be used that are monoclonal, preferably human or humanized, antibodies that have binding specificities for at least two different antigens. In the present case, one of the binding specificities is for at least one Mut-IL18 or Mut-IL-18R protein, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, e.g., in WO 93/08829, U.S. Pat. Nos., 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986), each entirely incorporated herein by reference.

Such antibodies optionally further affect a specific ligand, such as but not limited to where such antibody modulates, decreases, increases, antagonizes, angonizes, mitigates, aleviates, blocks, inhibits, abrogates and/or interferes with at least one Mut-IL18 or Mut-IL-18R activity or binding, or with Mut-IL18 or Mut-IL-18R receptor activity or binding, in vitro, in situ and/or in vivo. As a non-limiting example, a suitable Mut-IL18 or Mut-IL-18R antibody, specified portion or variant of the present invention can bind at least one Mut-IL18 or Mut-IL-18R, or specified portions, variants or domains thereof . A suitable Mut-IL18 or Mut-IL-18R antibody, specified portion, or variant can also optionally affect at least one of Mut-IL18 or Mut-IL-18R activity or function, such as but not limited to, RNA, DNA or protein synthesis, Mut-IL18 or Mut-IL-18R release, Mut-IL18 or Mut-IL-18R receptor signaling, membrane Mut-IL18 or Mut-IL-18R cleavage, Mut-IL18 or Mut-IL-18R activity, Mut-IL18 or Mut-IL-18R production and/or synthesis.

Mut-IL18 or Mut-IL-18R antibodies (also termed Mut-IL18 or Mut-IL-18R antibodies) useful in the methods and compositions of the present invention can optionally be characterized by high affinity binding to Mut-IL18 or Mut-IL-18R and optionally and preferably having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference).

Utility

The isolated nucleic acids of the present invention can be used for production of at least one Mut-IL18 or Mut-IL-18R antibody or specified variant thereof, which can be used to measure or effect in an cell, tissue, organ or animal (including mammals and humans), to diagnose, monitor, modulate, treat, alleviate, help prevent the incidence of, or reduce the symptoms of, at least one Mut-IL18 or Mut-IL-18R condition, selected from, but not limited to, at least one of an immune disorder or disease, a cardiovascular disorder or disease, an infectious, malignant, and/or neurologic disorder or disease, or other known or specified Mut-IL18 or Mut-IL-18R related condition.

Such a method can comprise administering an effective amount of a composition or a pharmaceutical composition comprising at least one Mut-IL18 or Mut-IL-18R antibody to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous adminstration, or any effective range or value therein, as done and determined using known methods, as described herein or known in the relevant arts.

Citations

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

### ANTIBODIES OF THE PRESENT INVENTION

At least one Mut-IL18 or Mut-IL-18R antibody of the present invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001), each entirely incorporated herein by reference.

Human antibodies that are specific for human Mut-IL18 or Mut-IL-18R proteins or fragments thereof can be raised against an appropriate immunogenic antigen, such as isolated and/or Mut-IL18 or Mut-IL-18R protein or a portion thereof (including synthetic molecules, such as synthetic peptides). Other specific or general mammalian antibodies can be similarly raised. Preparation of immunogenic antigens, and monoclonal antibody production can be performed using any suitable technique.

In one approach, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMAIWA, NEURO 2A, or the like, or heteromylomas, fusion products thereof, or any cell or fusion cell derived therefrom, or any other suitable cell line as known in the art. See, e.g., www.atcc.org, www.lifetech.com., and the like, with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Antibody producing cells can also be obtained from the peripheral blood or, preferably the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; Biolnvent, Lund, Sweden; Dyax Corp., Enzon, Affymax/Biosite; Xoma, Berkeley, Calif.; lxsys. See, e.g., EP 368,684, PCT/GB91/01134; PCT/GB92/01755; PCT/GB92/002240; PCT/GB92/00883; PCT/GB93/00605; U.S. Ser. No. 08/350260(May 12, 1994); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); EP 614 989 (MorphoSys); WO95/16027 (Biolnvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins U.S. Pat. Nos. 5,723,323, 5,763,192, 5,814,476, 5,817,483, 5,824,514, 5,976,862, WO 86/05803, EP 590 689 (Ixsys, now Applied Molecular Evolution (AME), each entirely incorporated herein by reference) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (Nov. 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B. V., Amsterdam, Netherlands (1988)).

Methods for engineering or humanizing non-human or human antibodies can also be used and are well known in the art. Generally, a humanized or engineered antibody has one or more amino acid residues from a source which is non-human, e.g., but not limited to mouse, rat, rabbit, non-human primate or other mammal. These human amino acid residues are often referred to as “import” residues, which are typically taken from an “import” variable, constant or other domain of a known human sequence. Known human Ig sequences are disclosed, e.g., www.ncbi.nlm.nih.gov/entrez/query.fcgi; www.atcc.org/phage/hdb.html; www.sciquest.com/; www.abcam.com/; www.antibodyresource.com/onlinecomp.html; www.public.iastate.edu/˜pedro/research_tools.html; www.mgen.uni-heidelberg.de/SD/IT/IT.html; www.whfreeman.com/immunology/CH05/kuby05.htm; www.library.thinkquest.org/12429/Immune/Antibody.html; www.hhmi.org/grants/lectures/1996/vlab/; www.path.cam.ac.uk/˜mrc7/mikeimages.html; www.antibodyresource.com/; mcb.harvard.edu/BioLinks/Immunology.html.www.immunologylink.com/; pathbox.wustl.edu/˜hcenter/index.html; www.biotech.ufl.edu/˜hcl/; www.pebio.com/pa/340913/340913.html; www.nal.usda.gov/awic/pubs/antibody/; www.m.ehime-u.ac.jp/˜yasuhito/Elisa.html; www.biodesign.com/table.asp; www.icnet.uk/axp/facs/davies/links.html; www.biotech.ufl.edu/˜fccl/protocol.html; www.isac-net.org/sites_geo.html; aximt1.imt.uni-marburg.de/˜rek/AEPStart.html; baserv.uci.kun.nl/˜jraats/linksl.html; www.recab.uni-hd.de/immuno.bme.nwu.edu/; www.mrc-cpe.cam.ac.uk/imt-doc/public/INTRO.html; www.ibt.unam.mx/vir/V_mice.html; imgt.cnusc.fr:8104/; www.biochem.ucl.ac.uk/˜martin/abs/index.html; antibody.bath.ac.uk/; abgen.cvm.tamu.edu/lab/wwwabgen.html; www.unizh.ch/˜honegger/AHOseminar/Slide01.html; www.cryst.bbk.ac.uk/˜ubcg07s/; www.nimr.mrc.ac.uk/CC/ccaewg/ccaewg.htm; www.path.cam.ac.uk/˜mrc7/humanisation/TAHHP.html; www.ibt.unam.mx/vir/structure/stat_aim.html; www.biosci.missouri.edu/smithgp/index.html; www.cryst.bioc.cam.ac.uk/˜fmolina/Web-pages/Pept/spottech.html; www.jerini.de/fr_products.htm; www.patents.ibm.com/ibm.html.Kabat et al., Sequences of Proteins of Immunological Interest, U.S. Dept. Health (1983), each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. Generally part or all of the non-human or human CDR sequences are maintained while the non-human sequences of the variable and constant regions are replaced with human or other amino acids. antibodies can also optionally be humanized with retention of high affinity for the antigen and other favorable biological properties. To achieve this goal, humanized antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos.: 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, PCT/: U.S. Ser. Nos. 98/16280, 96/18978, 91/09630, 91/05939, 94/01234, GB89/01334, GB91/01134, GB92/01755; WO90/14443, WO90/14424, WO90/14430, EP 229246, each entirely incorporated herein by reference, included references cited therein.

The Mut-IL18 or Mut-IL-18R antibody can also be optionally generated by immunization of a transgenic animal (e.g., mouse, rat, hamster, non-human primate, and the like) capable of producing a repertoire of human antibodies, as described herein and/or as known in the art. Cells that produce a human Mut-IL18 or Mut-IL-18R antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos: 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278. Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge antibody Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra, each of the above patents and publications entirely incorporated herein by reference.

Antibodies of the present invention can also be prepared using at least one Mut-IL18 or Mut-IL-18R antibody encoding nucleic acid to provide transgenic animals or mammals, such as goats, cows, horses, sheep, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies of the present invention can additionally be prepared using at least one Mut-IL18 or Mut-IL-18R antibody encoding nucleic acid to provide transgenic plants and cultured plant cells (e.g., but not limited to tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and reference cited therein. Thus, antibodies of the present invention can also be produced using transgenic plants, according to know methods. See also, e.g., Fischer et al., Biotechnol. Appl. Biochem. 30:99-108 (Oct., 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. See, also generally for plant expression of antibodies, but not limited to, Each of the above references is entirely incorporated herein by reference.

The antibodies of the invention can bind human Mut-IL18 or Mut-IL-18R with a wide range of affinities (K_{D}). In a preferred embodiment, at least one human mAb of the present invention can optionally bind human Mut-IL18 or Mut-IL-18R with high affinity. For example, a human mab can bind human Mut-IL18 or Mut-IL-18R with a K_{D} equal to or less than about 10⁻⁷ M, such as but not limited to, 0.1-9.9 (or any range or value therein) X 10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

Nucleic Acid Molecules

Using the information provided herein, such as the nucleotide sequences encoding at least 70-100% of the contiguous amino acids of at least one of SEQ ID NOS: 1-2, specified fragments, variants or consensus sequences thereof, or a deposited vector comprising at least one of these sequences, a nucleic acid molecule of the present invention encoding at least one Mut-IL18 or Mut-IL-18R antibody can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as theanti-sense strand.

Isolated nucleic acid molecules of the present invention can include nucleic acid molecules comprising an open reading frame (ORF), optionally with one or more introns, e.g., but not limited to, at least one specified portion of at least one CDR, as CDR1, CDR2 and/or CDR3 of at least one heavy chain or light chain; nucleic acid molecules comprising the coding sequence for an Mut-IL18 or Mut-IL-18R antibody or variable region; and nucleic acid molecules which comprise a nucleotide sequence substantially different from those described above but which, due to the degeneracy of the genetic code, still encode at least one Mut-IL18 or Mut-IL-18R antibody as described herein and/or as known in the art. Of course, the genetic code is well known in the art. Thus, it would be routine for one skilled in the art to generate such degenerate nucleic acid variants that code for specific Mut-IL18 or Mut-IL-18R antibodies of the present invention. See, e.g., Ausubel, et al., supra, and such nucleic acid variants are included in the present invention. Non-limiting examples of isolated nucleic acid molecules of the present inveniton include the CDR sequences corresponding to non-limiting examples of a nucleic acid encoding, respectively, HC CDR1, HC CDR2, HC CDR3, LC CDR1, LC CDR2, LC CDR3, HC variable region and LC variable region.

As indicated herein, nucleic acid molecules of the present invention which comprise a nucleic acid encoding an Mut-IL18 or Mut-IL-18R antibody can include, but are not limited to, those encoding the amino acid sequence of an antibody fragment, by itself; the coding sequence for the entire antibody or a portion thereof; the coding sequence for an antibody, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion.

Polynucleotides Which Selectively Hybridize to a Polynucleotide as Described Herein

The present invention provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably at least 85% or 90% full-length sequences, and more preferably at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides of this invention will encode at least a portion of an antibody encoded by the polynucleotides described herein. The polynucleotides of this invention embrace nucleic acid sequences that can be employed for selective hybridization to a polynucleotide encoding an antibody of the present invention. See, e.g., Ausubel, supra; Colligan, supra, each entirely incorporated herein by reference.

Construction of Nucleic Acids

The isolated nucleic acids of the present invention can be made using (a) recombinant methods, (b) synthetic techniques, (c) purification techniques, or combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention-excluding the coding sequence-is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

Recombinant Methods for Constructing Nucleic Acids

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, is well known to those of ordinary skill in the art. (See, e.g., Ausubel, supra; or Sambrook, supra)

Nucleic Acid Screening and Isolation Methods

A cDNA or genomic library can be screened using a probe based upon the sequence of a polynucleotide of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therem. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al,.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; 4,766,067 to Biswas; U.S. Pat. No. 4,656,134 to Ringold) and RNA mediated amplification that usesanti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boebringer Mannheim) can be used to improve yield of long PCR products.

Synthetic Methods for Constructing Nucleic Acids

The isolated nucleic acids of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

Recombinant Expression Cassettes

The present invention further provides recombinant expression cassettes comprising a nucleic acid of the present invention. A nucleic acid sequence of the present invention, for example a cDNA or a genomic sequence encoding an antibody of the present invention, can be used to construct a recombinant expression cassette that can be introduced into at least one desired host cell. A recombinant expression cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In some embodiments, isolated nucleic acids that serve as promoter, enhancer, or other elements can be introduced in the appropriate position (upstream, downstream or in intron) of a non-heterologous form of a polynucleotide of the present invention so as to up or down regulate expression of a polynucleotide of the present invention. For example, endogenous promoters can be altered in vivo or in vitro by mutation, deletion and/or substitution.

Vectors And Host Cells

The present invention also relates to vectors that include isolated nucleic acid molecules of the present invention, host cells that are genetically engineered with the recombinant vectors, and the production of at least one Mut-IL18 or Mut-IL-18R antibody by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one antibody of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the present invention.

Alternatively, nucleic acids of the present invention can be expressed in a host cell by turning on (by manipulation) in a host cell that contains endogenous DNA encoding an antibody of the present invention. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to an origin of replication; a promoter (e.g., late or early SV40 promoters, the CMV promoter (U.S. Pat. Nos. 5,168,062; 5,385,839), an HSV tk promoter, a pgk (phosphoglycerate kiinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

Purification of an Antibody

An Mut-IL18 or Mut-IL-18R antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affimty chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

Mut-IL18 or Mut-IL-18R Proteins and Antibodies

The isolated proteins and antibodies of the present invention comprise at least one protein and/or antibody amino acid sequence disclosed or described herein encoded by any suitable polynucleotide, or any at least one isolated or prepared protein antibody. Preferably, the at least one protein has at least one Mut-IL18 or Mut-IL-18R activity and the at least one antibody binds human Mut-IL18 or Mut-IL-18R and, thereby partially or substantially modulates at least one structural or biological activity of at least one Mut-IL18 or Mut-IL-18R protein.

As used herein, the term “Mut-IL18 or Mut-L-18R protein” refers to a protein as described herein that has at least one Mut-IL18 or Mut-IL-18R-dependent activity, such as 5-10000%, of the activity of a known or other Mut-IL18 or Mut-L-18R protein or active portion thereof, preferably by at least about 10, 20, 30, 40, 50, 55, 60, 65, 70, 75, 80, 85, 90, 91, 92, 93, 94, 95, 96, 97, 98, 99, 100% or more, depending on the assay. The capacity of a Mut-IL18 or Mut-IL-18R protein to have at least one Mut-IL18 or Mut-IL-18R-dependent activity is preferably assessed by at least one suitable Mut-IL18 or Mut-L-18R protein or receptor assay, as described herein and/or as known in the art.

As used herein, the term “neutralizing antibody” refers to an antibody that can inhibit at least one Mut-IL18 or Mut-IL-18R-dependent activity by about 5-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an Mut-IL18 or Mut-IL-18R antibody to inhibit an Mut-L18 or Mut-IL-18R-dependent activity is preferably assessed by at least one suitable Mut-IL18 or Mut-IL-18R protein or receptor assay, as described herein and/or as known in the art. An antibody of the invention can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4. Antibodies of this type can be prepared by employing a transgenic mouse or other trangenic non-human mammal comprising at least one human light chain (e.g., IgG, IgA and IgM (e.g., γ1, γ2, γ3, γ4) transgenes as described herein and/or as known in the art. In another embodiment, the human Mut-IL18 or Mut-IL-18R human antibody comprises an IgG1 heavy chain and a IgG1 light chain.

At least one antibody of the invention binds at least one specified epitope specific to at least one Mut-IL18 or Mut-IL-18R protein, subunit, fragment, portion or any combination thereof. The at least one epitope can comprise at least one antibody binding region that comprises at least one portion of the protein, which epitope can optionally comprise at least one portion of at least one extracellular, soluble, hydrophillic, external or cytoplasmic portion of the protein. The at least one specified epitope can comprise any combination of at least one amino acid sequence of at least 1-3 amino acids to the entire specified portion of contiguous amino acids of the SEQ ID NOS: 1-2.

The at least one antibody of the present invention can preferably comprise at least one antigen-binding region that comprises at least one human complementarity determining region (CDR1, CDR2 and CDR3) or variant of at least one heavy chain variable region and/or at least one human complementarity determining region (CDR1, CDR2 and CDR3) or variant of at least one light chain variable region. In a particular embodiment, the protein and antibody can have an antigen-binding region that comprises at least a portion of at least one heavy chain (HC) CDR (i.e., HC CDR1, HC CDR2 and/or HC CDR3) having the amino acid sequence of the corresponding HC CDRs 1, 2 and/or 3. In another particular embodiment, the antibody or antigen-binding portion or variant can have at least one antigen-binding region that comprises at least a portion of at least one light chain (LC) CDR (i.e., LC CDR1, LC CDR2 and/or LC CDR3). In a preferred embodiment the three heavy chain CDRs and the three light chain CDRs of the anitbody or antigen-binding fragment have the amino acid sequence of the corresponding CDR of at least one of mAb , , , as described herein. Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

The Mut-IL18 or Mut-IL-18R antibody can comprise at least one of a heavy or light chain variable region having a defined amino acid sequence. For example, in a preferred embodiment, the Mut-IL18 or Mut-IL-18R antibody comprises at least one of at least one heavy chain variable region; and/or at least one light chain variable region. Antibodies that bind to human Mut-IL18 or Mut-IL-18R and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human Mut-IL18 or Mut-IL-18R or a fragment thereof to elicit the production of antibodies. If desired, the antibody producing cells can be isolated and hybridomas or other immortalized antibody-producing cells can be prepared as described herein and/or as known in the art. Alternatively, the antibody, specified portion or variant can be expressed using the encoding nucleic acid or portion thereof in a suitable host cell.

The invention also relates to antibodies, antigen-binding fragments, immunoglobulin chains and CDRs comprising amino acids in a sequence that is substantially the same as an amino acid sequence described herein. Preferably, such antibodies or antigen-binding fragments and antibodies comprising such chains or CDRs can bind human Mut-IL18 or Mut-IL-18R with high affinity (e.g., K_{D} less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g, charge, structure, polarity, hydrophobicity/ hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and gfycine (G); F, W and Y; C, S and T.

Amino Acid Codes

The amino acids that make up Mut-IL18 or Mut-IL-18R antibodies of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994):

| SINGLE | THREE | | |
|---|---|---|---|
| LETTER | LETTER | | THREE NUCLEOTIDE |
| CODE | CODE | NAME | CODON(S) |
| A | Ala | Alanine | GCA, GCC, GCG, GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, CUC, |
| | | | CUG, CUU |
| M | Met | Methionine | AUG |
| N | Asn | Asparagine | AAC, AAU |
| P | Pro | Proline | CCA, CCC, CCG, CCU |
| Q | Gln | Glutamine | CAA, CAG |
| R | Arg | Arginine | AGA, AGG, CGA, CGC, |
| | | | CGG, CGU |
| S | Ser | Serine | AGC, AGU, UCA, UCC, |
| | | | UCG, UCU |
| T | Thr | Threonine | ACA, ACC, ACG, ACU |
| V | Val | Valine | GUA, GUC, GUG, GUU |
| W | Trp | Tryptophan | UGG |
| Y | Tyr | Tyrosine | UAC, UAU |

An Mut-IL18 or Mut-IL-18R antibody of the present invention can include one or more amino acid substitutions, deletions or additions, either from natural mutations or human manipulation, as specified herein.

Of course, the number of amino acid substitutions a skilled artisan would make depends on many factors, including those described above. Generally speaking, the number of amino acid substitutions, insertions or deletions for any given Mut-IL18 or Mut-IL-18R antibody, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an Mut-IL18 or Mut-IL-18R antibody of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to at least one Mut-IL18 or Mut-IL-18R neutralizing activity. Sites that are critical for antibody binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

Mut-IL18 or Mut-IL-18R proteins of the present invention can include, but are not limited to, at least one portion, sequence or combination selected from 3-100 to all of the contiguous amino acids of at least one of SEQ ID NOS: 1-2.

Non-limiting CDRs or portions of Mut-IL18 or Mut-IL-18R proteins or antibodies of the invention that can enhance or maintain at least one of the listed activities include, but are not limited to, any of the above polypeptides, further comprising at least one mutation corresponding to at least one substitution selected from the group consisting of at least one of extracellular, intracellular, soluble, at least 10 contiguous amino acids, and the like, extracellular, intracellular, soluble, at least 10 contiguous amino acids, and the like, , , , , and/or.

Non-limiting variants that can enhance or maintain at least one of the listed activities include, but are not limited to, any of the above polypeptides, further comprising at least one mutation corresponding to at least one substitution selected from the group consisting of: Thr10 for Ser10; Val12 for Ile12; Ser45 for Thr45; Tyr47 for Phe47; Phe52 for Tyr52; Val64 for Ile64; Tyr101 for Phe101; Val5 for Leu5; Val20 for Leu20; Ile20 for Leu20; Tyr21 for Phe2l; Val22 for Ile22; Ile66 for Val66; Thr72 for Ser72; Phe148 for Ser148; Glu4 for Lys4; Ile6 for Glu6; Asp8 for Lys8; Ile13 for Argl3; Arg15 for Leu15; Lys17 for Asp17; Lys27 for Arg27; Ala30 for Phe30; Lys35 for Asp35; Phe37 for Asp37; Glu38 for Cys38; Ala39 for Arg39; Trp40 for Asp40; Glu51 for Met51; Gly53 for Lys53; Ile56 for Gln56; Ala58 for Arg58; Lys62 for Val62; Lys94 for Asp94; Phe95 for Thr95; Leu104 for Arg104; Ile108 for Gly108; Lys111 for Asn111; Phe129 for Lys129; Asp131 for Arg131; Leu132 for Asp132; Glu133 for Leu133; Ala134 for Phe134; Thr150 for Met150; Ser151 for Phe115, of at least one of SEQ ID NOS: 1-2.

A(n) Mut-IL18 or Mut-IL-18R protein can further optionally comprise a polypeptide of at least one of 70-100% of the contiguous amino acids of at least one of SEQ ID NOS: 1-2 or any variant thereof.

In one embodiment, the amino acid sequence of a Mut-IL18 or Mut-IL-18R protein or antibody has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of SEQ ID NOS: 1-2. Preferably, 70-100% amino acid identity (i.e., 90, 91, 92, 93, 94, 95, 96, 97, 98, 99, 100 or any range or value therein) is determined using a suitable computer algorithm, as known in the art.

The proteins and antibodies of the present invention, or specified variants thereof, can comprise any number of contiguous amino acid residues from an antibody of the present invention, wherein that number is selected from the group of integers consisting of from 10-100% of the number of contiguous residues in an Mut-IL18 or Mut-IL-18R protein or antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active protein or antibody of the present invention. Biologically active proteins or antibodies have a specific activity at least 20%, 30%, or 40%, and preferably at least 50%, 60%, or 70%, and most preferably at least 80%, 90%, or 95%-1000% of that of the native (non-synthetic), endogenous or related and known protein or antibody. Methods of assaying and quantifying measures of enzymatic activity and substrate specificity, are well known to those of skill in the art.

In another aspect, the invention relates to Mut-IL18 or Mut-IL-18R proteins or antibodies of the invention, as described herein, which are modified by the covalent attachment of a moiety. Such modification can produce a Mut-IL18 or Mut-IL-18R protein or anibody with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified proteins and antibodies of the invention can comprise one or more organic moieties that are covalently bonded, directly or indirectly, to the antibody or protein. Each organic moiety that is bonded to the protein or antibody of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, a Mut-IL18 or Mut-IL-18R antibody or protein modified by the covalent attachment of polylysine is encompassed by the invention. Hydrophilic polymers suitable for modifying antibodies or proteins of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the protein or antibody of the invention has a molecular weight of about 800 to about 150,000 Daltons as a separate molecular entity. For example PEG₅₀₀₀ and PEG_{20,000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), Cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human proteins and antibodies can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl)carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See, for example, Thompson, et al., WO 92/16221 the entire teachings of which are incorporated herein by reference.)

Modified proteins or antibodies of the invention can be produced by reacting the protein or antibody with a modifying agent. For example, the organic moieties can be bonded to the antibody or protein in a non-site specific manner by employing an amine-reactive modifying agent, for example, an NHS ester of PEG. Modified Mut-IL18 or Mut-IL-18R proteins or antibodies can also be prepared by reducing disulfide bonds (e.g., intra-chain disulfide bonds) of the protein and antibody. The reduced protein and antibody can then be reacted with a thiol-reactive modifying agent to produce the modified antibody of the invention. Modified proteins and antibodies comprising an organic moiety that is bonded to specific sites of an antibody of the present invention can be prepared using suitable methods, such as reverse proteolysis (Fisch el al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

IDIOTYPE ANTIBODIES TO Mut-IL18 or Mut-IL-18R ANTIBODY COMPOSITIONS

In addition to monoclonal or chimeric Mut-IL18 or Mut-IL-18R antibodies, the present invention is also directed to an idiotypic (Id) antibody specific for such antibodies of the invention. An anti-Id antibody is an antibody that recognizes unique determinants generally associated with the antigen-binding region of another antibody. The Id can be prepared by immunizing an animal of the same species and genetic type (e.g. mouse strain) as the source of the Id antibody with the antibody or a CDR containing region thereof. The immunized animal will recognize and respond to the idiotypic determinants of the immunizing antibody and produce an anti-Id antibody. The anti-Id antibody may also be used as an “immunogen” to induce an immune response in yet another animal, producing a so-called anti-Id antibody.

Mut-IL18 or Mut-IL-18R PROTEIN AND ANTIBODY COMPOSITIONS

The present invention also provides at least one Mut-IL18 or Mut-IL-18R antibody or protein composition comprising at least one, at least two, at least three, at least four, at least five, at least six or more Mut-IL18 or Mut-IL-18R antibodies or proteins thereof, as described herein and/or as known in the art that are provided in a non-naturally occurring composition, mixture or form. Such compositions comprise non-naturally occurring compositions comprising at least one or two Mut-IL18 or Mut-IL-18R antibody or protein amino acid sequences selected from the group consisting of 5-100% of the contiguous amino acids of SEQ ID NOS: 1-2, or specified fragments, domains or variants thereof. Further preferred compositions comprise 40-99% of at least one of 70-100% of SEQ ID NOS: 1-2, or specified fragments, domains or variants thereof. Such composition percentages are by weight, volume, concentration, molarity, or molality as liquid or dry solutions, mixtures, suspension, emulsions or colloids, as known in the art or as described herein.

Mut-IL18 or Mut-IL-18R antibody or protein compositions of the present invention can further comprise at least one of any suitable and effective amount of a composition or pharmaceutical composition comprising at least one Mut-IL18 or Mut-IL-18R antibody to a cell, tissue, organ, animal or patient in need of such modulation, treatment or therapy, optionally further comprising at least one selected from at least one TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Non-limiting examples of such cytokines include, but are not limted to, any of IL-1 to IL-23. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Such compositions can also include toxin molecules that are associated, bound, co-formulated or co-administered with at least one antibody or protein of the present invention. The toxin can optionally act to selectively kill the pathologic cell or tissue. The pathologic cell can be a cancer or other cell. Such toxins can be, but are not limited to, purified or recombinant toxin or toxin fragment comprising at least one functional cytotoxic domain of toxin, e.g., selected from at least one of ricin, diphtheria toxin, a venom toxin, or a bacterial toxin. The term toxin also includes both endotoxins and exotoxins produced by any naturally occurring, mutant or recombinant bacteria or viruses which may cause any pathological condition in humans and other mammals, including toxin shock, which can result in death. Such toxins may include, but are not limited to, enterotoxigenic E. coli heat-labile enterotoxin (LT), heat-stable enterotoxin (ST), Shigella cytotoxin, Aeromonas enterotoxins, toxic shock syndrome toxin-1 (TSST-1), Staphylococcal enterotoxin A (SEA), B (SEB), or C (SEC), Streptococcal enterotoxins and the like. Such bacteria include, but are not limited to, strains of a species of enterotoxigenic E. coli (ETEC), enterohemorrhagic E. coli (e.g., strains of serotype 0157:H7), Staphylococcus species (e.g., Staphylococcus aureus, Staphylococcus pyogenes), Shigella species (e.g., Shigella dysenteriae, Shigellaflexneri, Shigella boydi, and Shigella sonnei), Salmonella species (e.g., Salmonella typhi, Salmonella cholera-suis, Salmonella enteritidis), Clostridium species (e.g., Clostridium perfringens, Clostridium dificile, Clostridium botulinum), Camphlobacter species (e.g., Camphlobacterjejuni, Camphlobacterfetus), Heliobacter species, (e.g., Heliobacter pylori), Aeromonas species (e.g., Aeromonas sobria, Aeromonas hydrophila, Aeromonas caviae), Pleisomonas shigelloides, Yersina enterocolitica, Vibrios species (e.g., Vibrios cholerae, Vibrios parahemolyticus), Klebsiella species, Pseudomonas aeruginosa, and Streptococci. See, e.g., Stein, ed., INTERNAL MEDICINE, 3rd ed., pp 1-13, Little, Brown and Co., Boston, (1990); Evans et al., eds., Bacterial Infections of Humans: Epidemiology and Control, 2d. Ed., pp 239-254, Plenum Medical Book Co., New York (1991); Mandell et al, Principles and Practice of Infectious Diseases, 3d. Ed., Churchill Livingstone, N.Y. (1990); Berkow et al, eds., The Merck Manual, 16th edition, Merck and Co., Rahway, N.J., 1992; Wood et al, FEMS Microbiology Immunology, 76:121-134 (1991); Marrack et al, Science, 248:705-711 (1990), the contents of which references are incorporated entirely herein by reference.

Mut-IL18 or Mut-IL-18R antibody or protein compounds, compositions or combinations of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington 's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the Mut-IL18 or Mut-IL-18R antibody or protein composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary but non-limiting protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Mut-IL18 or Mut-IL-18R antibody or protein compositions can also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts such as citrate.

Additionally, Mut-IL 18 or Mut-IL-18R antibody or protein compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the Mut-IL18 or Mut-IL-18R antibody or protein compositions according to the invention are known in the art, e.g., as listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), the disclosures of which are entirely incorporated herein by reference. Preferrred carrier or excipient materials are carbohydrates (e.g., saccharides and alditols) and buffers (e.g., citrate) or polymeric agents.

Formulations

As noted above, the invention provides for stable formulations, which is preferably a phosphate buffer with saline or a chosen salt, as well as preserved solutions and formulations containing a preservative as well as multi-use preserved formulations suitable for pharmaceutical or veterinary use, comprising at least one Mut-IL18 or Mut-IL-18R antibody or protein in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1., 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one Mut-IL18 or Mut-IL-18R antibody or protein with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one Mut-IL18 or Mut-IL-18R antibody or protein, and a second vial comprising an aqueous diluent of prescribed buffer or preservative, wherein said packaging material comprises a label that instructs a patient to reconstitute the at least one Mut-IL18 or Mut-IL-18R antibody or protein in the aqueous diluent to form a solution that can be held over a period of twenty-four hours or greater.

The at least one Mut-IL18 or Mut-IL-18Rantibody or protein used in accordance with the present invention can be produced by recombinant means, including from mammalian cell or transgenic preparations, or can be purified from other biological sources, as described herein or as known in the art.

The range of at least one Mut-IL18 or Mut-IL-18R antibody in at least one product of the present invention includes amounts yielding upon reconstitution, if in a wet/dry system, concentrations from about 1.0 ng/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

The range of at least one Mut-IL18 or Mut-IL-18R antibody in at least one product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chlonde, sodium dehydroacetate and thimerosal, or mixtures thereof. The concentration of preservative used in the formulation is a concentration sufficient to yield an microbial effect. Such concentrations are dependent on the preservative selected and are readily determined by the skilled artisan.

Other excipients, e.g. isotonicity agents, buffers, antioxidants, preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably the formulations of the present invention have pH between about 6.8 and about 7.8. Preferred buffers include phosphate buffers, most preferably sodium phosphate, particularly phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one Mut-IL18 or Mut-IL-18R antibody or protein and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one Mut-IL18 or Mut-IL-18R antibody or protein and preservative in an aqueous diluent is carried out using conventional dissolution and mixing procedures. To prepare a suitable formulation, for example, a measured amount of at least one Mut-IL18 or Mut-IL-18R antibody or protein in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed formulations can be provided to patients as clear solutions or as dual vials comprising a vial of lyophilized at least one Mut-IL18 or Mut-IL-18R antibody or protein that is reconstituted with a second vial containing water, a preservative and/or excipients, preferably a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period of immediately to twenty-four hours or greater. Accordingly, the presently claimed articles of manufacture offer significant advantages to the patient. Formulations of the invention can optionally be safely stored at temperatures of from about 2 to about 40° C. and retain the biologically activity of the protein for extended periods of time, thus, allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of at least one Mut-IL18 or Mut-IL-18R antibody or protein in the invention can be prepared by a process that comprises mixing at least one antibody or protein in an aqueous diluent. Mixing is carried out using conventional dissolution and mixing procedures. To prepare a suitable diluent, for example, a measured amount of at least one antibody or protein in water or buffer is combined in quantities sufficient to provide the protein and optionally a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one Mut-IL18 or Mut-IL-18R antibody or protein that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one Mut-IL18 or Mut-IL-18R antibody or protein that is reconstituted with a second vial containing the aqueous diluent. The clear solution in this case can be up to one liter or even larger in size, providing a large reservoir from which smaller portions of the at least one antibody or protein solution can be retrieved one or multiple times for transfer into smaller vials and provided by the pharmacy or clinic to their customers and/or patients.

Recognized devices comprising these single vial systems include those pen-injector devices for delivery of a solution such as BD Pens, BD Autojector®, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, iject®, J-tip Needle-Free Injector®, Intraject®, Medi-Ject®, e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com). Recognized devices comprising a dual vial system include those pen-injector systems for reconstituting a lyophilized drug in a cartridge for delivery of the reconstituted solution such as the HumatroPen®.

The products presently claimed include packaging material. The packaging material provides, in addition to the information required by the regulatory agencies, the conditions under which the product can be used. The packaging material of the present invention provides instructions to the patient to reconstitute the at least one Mut-IL18 or Mut-IL-18R antibody or protein in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one Mut-IL18 or Mut-IL-18R antibody or protein and a selected buffer, preferably a phosphate buffer containing saline or a chosen salt. Mixing the at least one antibody or protein and buffer in an aqueous diluent is carried out using conventional dissolution and mixing procedures. To prepare a suitable formulation, for example, a measured amount of at least one antibody or protein in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized at least one Mut-IL18 or Mut-IL-18R antibody or protein that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

At least one Mut-IL18 or Mut-IL-18R antibody or protein in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

Therapeutic Applications

The present invention also provides a method for modulating or treating at least one Mut-IL18 or Mut-IL-18R related disease, in a cell, tissue, organ, animal, or patient, as known in the art or as described herein, using at least one antibody or protein of the present invention.

The present invention also provides a method for modulating or treating at least one Mut-IL18 or Mut-IL-18R related disease, in a cell, tissue, organ, animal, or patient including, but not limited to, at least one of obesity, an immune related disease, a cardiovascular disease, an infectious disease, a malignant disease or a neurologic disease.

The present invention also provides a method for modulating or treating at least one adult or pediatric immune or inflammation related disease, in a cell, tissue, organ, animal, or patient including, but not limited to, at least one of, or at least one inflammation related to, rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, Crohn's disease, systemic lupus erythematosis, antiphospholipid syndrome, iridocyclitis, uveitis, optic neuritis, idiopathic pulmonary fibrosis, systemic vasculitis, Wegener's granulomatosis, sarcoidosis, orchitis, vasectomy or vasectomy reversal procedures, allergic atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma, hemorrhage, burns, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, type I or type II diabetes, nephrosis, atopic diseases, hypersensitity reactions, allergic rhinitis, hay fever, perennial rhinitis, conjunctivitis, endometriosis, asthma, urticaria, systemic anaphalaxis, dermatitis, pernicious anemia, hemolytic disesease, thrombocytopenia, graft rejection of any organ or tissue, kidney translplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, receptor hypersensitivity reactions, chronic obstructive pulmonary disease (COPD), Graves disease, Raynoud's disease, type B insulin-resistant diabetes, asthma, myasthenia gravis, antibody-meditated cytotoxicity, gene therapy inflammation (e.g., adenovirus, AAV, vaccinia, DNA or RNA, Muloney murine leukemia virus (MMLV) and the like), type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, diabetes mellitus, chronic active hepatitis, primary billiary cirrhosis, vitiligo, vasculitis, post-MI cardiotomy syndrome, type IV hypersensitivity , contact dermatitis, hypersensitivity pneumonitis, allograft rejection, granulomas due to intracellular organisms, drug sensitivity, metabolic, idiopathic, Wilson's disease, hemachromatosis, alpha-l-antitrypsin deficiency, diabetic retinopathy, Hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, okt3 therapy, cd3 therapy, cytokine therapy, chemotherapy, radiation therapy (e.g., including but not limited toasthenia, anemia, cachexia, and the like), chronic salicylate intoxication, and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2000), each entirely incorporated by reference.

The present invention also provides a method for modulating or treating at least one cardiovascular disease in a cell, tissue, organ, animal, or patient, including, but not limited to, at least one of cardiac stun syndrome, myocardial infarction, congestive heart failure, stroke, ischemic stroke, hemorrhage, arteriosclerosis, atherosclerosis, restenosis, diabetic ateriosclerotic disease, hypertension, arterial hypertension, renovascular hypertension, syncope, shock, syphilis of the cardiovascular system, heart failure, cor pulmonale, primary pulmonary hypertension, cardiac arrhythmias, atrial ectopic beats, atrial flutter, atrial fibrillation (sustained or paroxysmal), post perfusion syndrome, cardiopulmonary bypass inflammation response, chaotic or multifocal atrial tachycardia, regular narrow QRS tachycardia, specific arrythmias, ventricular fibrillation, His bundle arrythmias, atrioventricular block, bundle branch block, myocardial ischemic disorders, coronary artery disease, angina pectoris, myocardial infarction, cardiomyopathy, dilated congestive cardiomyopathy, restrictive cardiomyopathy, valvular heart diseases, endocarditis, pericardial disease, cardiac tumors, aordic and peripheral aneuryisms, aortic dissection, inflammation of the aorta, occulsion of the abdominal aorta and its branches, peripheral vascular disorders, occulsive arterial disorders, peripheral atherlosclerotic disease, thromboangitis obliterans, functional peripheral arterial disorders, Raynaud's phenomenon and disease, acrocyanosis, erythromelalgia, venous diseases, venous thrombosis, varicose veins, arteriovenous fistula, lymphederma, lipedema, unstable angina, reperfusion injury, post pump syndrome, ischemia-reperfusion injury, and the like. Such a method can optionally comprise administering an effective amount of a composition or pharmaceutical composition comprising at least one Mut-IL18 or Mut-IL-18R antibody or protein to a cell, tissue, organ, animal or patient in need of such modulation, treatment or therapy.

The present invention also provides a method for modulating or treating at least one infectious disease in a cell, tissue, organ, animal or patient, including, but not limited to, at least one of: acute or chronic infection, acute and chronic parasitic or infectious processes, including bacterial, viral and fungal infections, HIV infection, HIV neuropathy, meningitis, hepatitis (A,B or C, or the like), septic arthritis, peritonitis, pneumonia, epiglottitis, e. coli 0157:h7, hemolytic uremic syndrome, thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis, epidydimitis, legionella, lyme disease, influenza a, epstein-barr virus, vital-associated hemaphagocytic syndrome, vital encephalitis, aseptic meningitis, and the like. Such toxins can be, but are not limited to, purified or recombinant toxin or toxin fragment comprising at least one functional cytotoxic domain of toxin, e.g., selected from at least one of diphtheria toxin, a venom toxin, a viral toxin or a bacterial toxin. The term toxin also includes both endotoxins and exotoxins produced by any naturally occurring, mutant or recombinant bacteria or viruses which may cause any pathological condition in humans and other mammals, including toxin shock, which can result in death. Such toxins may include, but are not limited to, enterotoxigenic E. coli heat-labile enterotoxin (LT), heat-stable enterotoxin (ST), Shigella cytotoxin, Aeromonas enterotoxins, toxic shock syndrome toxin-1 (TSST-1), Staphylococcal enterotoxin A (SEA), B (SEB), or C (SEC), Streptococcal enterotoxins anthrax endotoxin, and the like. Such bacteria include, but are not limited to, gram negative or gram positive bactieria, Bacillus, E. coli, Streptococcus, Staphlococcus, Shigella, Salmonella, Clostridium, Camphbacter, Heliobacter, Aeromonas, Enteroccis, Pseudomonas, and the like, such as but not limited to, strains of a species of enterotoxigenic E. coli (ETEC), enterohemorrhagic E. coli (e.g., strains of serotype 0157:H7), Staphylococcus species (e.g., Staphylococcus aureus, Staphylococcus pyogenes), Shigella species (e.g., Shigella dysenteriae, Shigellaflexneri, Shigella boydii, and Shigella sonnei), Salmonella species (e.g., Salmonella typhi, Salmonella cholera-suis, Salmonella enteritidis), Clostridium species (e.g., Clostridium perfringens, Clostridium dificile, Clostridium botulinum), Camphlobacter species (e.g., Camphlobacter jejuni, Camphlobacter fetus), Heliobacter species, (e.g., Heliobacter pylori), Aeromonas species (e.g., Aeromonas sobria, Aeromonas hydrophila, Aeromonas caviae), Pleisomonas shigelloides, Yersina enterocolitica, Vibrios species (e.g., Vibrios cholerae, Vibrios parahemolyticus), Klebsiella species, Pseudomonas aeruginosa, and Streptococci. See, e.g., Stein, ed., INTERNAL MEDICINE, 3rd ed., pp 1-13, Little, Brown and Co., Boston, (1990); Evans et al., eds., Bacterial Infections of Humans: Epidemiology and Control, 2d. Ed., pp 239-254, Plenum Medical Book Co., New York (1991); Mandell et al, Principles and Practice of Infectious Diseases, 3d. Ed., Churchill Livingstone, N.Y. (1990); Berkow et al, eds., The Merck Manual, 16th edition, Merck and Co., Rahway, N.J., 1992; Wood et al, FEMS Microbiology Immunology, 76:121-134 (1991); Marrack et al, Science, 248:705-711 (1990), the contents of which references are incorporated entirely herein by reference. Such a method can optionally comprise administering an effective amount of a composition or pharmaceutical composition comprising at least one Mut-IL18 or Mut-IL-18R antibody or protein to a cell, tissue, organ, animal or patient in need of such modulation, treatment or therapy.

The present invention also provides a method for modulating or treating at least one malignant disease in a cell, tissue, organ, animal or patient, including, but not limited to, at least one of: leukemia, acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia, myelodyplastic syndrome (MDS), a lymphoma, Hodgkin's disease, a malignamt lymphoma, non-hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome, hypercalcemia of malignancy, solid tumors, CD-46 related tumors, adenocarcinomas, sarcomas, malignant melanoma, hemangioma, metastatic disease, cancer related bone resorption, cancer related bone pain, and the like. Such a method can optionally comprise administering an effective amount of a composition or pharmaceutical composition comprising at least one Mut-IL18 or Mut-IL-18R antibody or protein to a cell, tissue, organ, animal or patient in need of such modulation, treatment or therapy.

The present invention also provides a method for modulating or treating at least one neurologic disease in a cell, tissue, organ, animal or patient, including, but not limited to, at least one of: neurodegenerative diseases, multiple sclerosis, migraine headache, AIDS dementia complex, demyelinating diseases, such as multiple sclerosis and acute transverse myelitis; extrapyramidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supranucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi.system disorder); demyelinating core disorders, such as multiple sclerosis, acute transverse myelitis; and disorders of the motor unit such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Hallerrorden-Spatz disease; and Dementia pugilistica, and the like. Such a method can optionally comprise administering an effective amount of a composition or pharmaceutical composition comprising at least one Mut-IL18 or Mut-IL-18R antibody or protein to a cell, tissue, organ, animal or patient in need of such modulation, treatment or therapy. See, e.g., the Merck Manual, 16^{th} Edition, Merck & Company, Rahway, N.J. (1992)

Any method of the present invention can comprise administering an effective amount of a composition or pharmaceutical composition comprising at least one Mut-IL18 or Mut-IL-18R antibody or protein to a cell, tissue, organ, animal or patient in need of such modulation, treatment or therapy. Such a method can optionally further comprise co-administration or combination therapy for treating such diseases, wherein the administering of said at least one Mut-IL18 or Mut-IL-18R antibody or protein, specified portion or variant thereof, further comprises administering, before concurrently, and/or after, at least one selected from at least one TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsonatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

TNF antagonists suitable for compositions, combination therapy, co-administration, devices and/or methods of the present invention (further comprising at least one anti body, specified portion and variant thereof, of the present invention), include, but are not limited to, TNF antibodies, antigen-binding fragments thereof, and receptor molecules which bind specifically to TNF; compounds which prevent and/or inhibit TNF synthesis, TNF release or its action on target cells, such as thalidomide, tenidap, phosphodiesterase inhibitors (e.g, pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

As used herein, a “tumor necrosis factor antibody,” “TNF antibody,” “TNFα antibody,” or fragment and the like decreases, blocks, inhibits, abrogates or interferes with TNFα activity in vitro, in situ and/or preferably in vivo. For example, a suitable TNF human antibody of the present invention can bind TNFα and includes TNF antibodies, antigen-binding fragments thereof, and specified mutants or domains thereof that bind specifically to TNFα. A suitable TNF anttibody or fragment can also decrease block, abrogate, interfere, prevent and/or inhibit TNF RNA, DNA or protein synthesis, TNF release, TNF receptor signaling, membrane TNF cleavage, TNF activity, TNF production and/or synthesis.

Chimeric antibody cA2 consists of the antigen binding variable region of the high-affinity neutralizing mouse human TNFα IgG1 antibody, designated A2, and the constant regions of a human IgG1, kappa immunoglobulin. The human IgG1 Fc region improves allogeneic antibody effector function, increases the circulating serum half-life and decreases the immunogenicity of the antibody. The avidity and epitope specificity of the chimeric antibody cA2 is derived from the variable region of the murine antibody A2. In a particular embodiment, a preferred source for nucleic acids encoding the variable region of the murine antibody A2 is the A2 hybridoma cell line.

Chimeric A2 (cA2) neutralizes the cytotoxic effect of both natural and recombinant human TNFα in a dose dependent manner. From binding assays of chimeric antibody cA2 and recombinant human TNFα, the affinity constant of chimeric antibody cA2 was calculated to be 1.04×10¹⁰M⁻¹. Preferred methods for determining monoclonal antibody specificity and affinity by competitive inhibition can be found in Harlow, et al., antibodies: A Laboratory Manual, Cold Spring Harbor Laboratory Press, Cold Spring Harbor, N.Y., 1988; Colligan et al., eds., Current Protocols in Immunology, Greene Publishing Assoc. and Wiley Interscience, New York, (1992-2000); Kozbor et al., Immunol. Today, 4:72-79 (1983); Ausubel et al., eds. Current Protocols in Molecular Biology, Wiley Interscience, New York (1987-2000); and Muller, Meth. Enzymol., 92:589-601 (1983), which references are entirely incorporated herein by reference.

In a particular embodiment, murine monoclonal antibody A2 is produced by a cell line designated cl34A. Chimeric antibody cA2 is produced by a cell line designated c168A.

Additional examples of monoclonal TNF antibodies that can be used in the present invention are described in the art (see, e.g., U.S. Pat. No. 5,231,024; Möller, A. et al., Cytokine 2(3): 162-169 (1990); U.S. application Ser. No. 07/943,852 (filed Sep. 11, 1992); Rathjen et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); and Hirai, et al., J. Immunol. Meth. 96:57-62 (1987), which references are entirely incorporated herein by reference).

TNF Receptor Molecules

Preferred TNF receptor molecules useful in the present invention are those that bind TNFα with high affinity (see, e.g., Feldmann et al., International Publication No. WO 92/07076 (published Apr. 30, 1992); Schall et al., Cell 61:361-370 (1990); and Loetscher et al., Cell 61:351-359 (1990), which references are entirely incorporated herein by reference) and optionally possess low immunogenicity. In particular, the 55 kDa (p55 TNF-R) and the 75 kDa (p75 TNF-R) TNF cell surface receptors are useful in the present invention. Truncated forms of these receptors, comprising the extracellular domains (ECD) of the receptors or functional portions thereof (see, e.g., Corcoran et al., Eur. J. Biochem. 223:831-840 (1994)), are also useful in the present invention. Truncated forms of the TNF receptors, comprising the ECD, have been detected in urine and serum as 30 kDa and 40 kDa TNFα inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof, are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high affinity, as well as other undefined properties, can contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. An example of such a TNF immunoreceptor fusion molecule is TNF receptor/IgG fusion protein. TNF immunoreceptor fusion molecules and methods for their production have been described in the art (Lesslauer et al., Eur. J. Immunol. 21:2883-2886 (1991); Ashkenazi et al., Proc. Natl. Acad. Sci. USA 88:10535-10539 (1991); Peppel et al., J. Exp. Med. 174:1483-1489 (1991); Kolls et al., Proc. Natl. Acad. Sci. USA 91:215-219 (1994); Butler et al., Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (filed May 16, 1995), each of which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

A functional equivalent, derivative, fragment or region of TNF receptor molecule refers to the portion of the TNF receptor molecule, or the portion of the TNF receptor molecule sequence which encodes TNF receptor molecule, that is of sufficient size and sequences to functionally resemble TNF receptor molecules that can be used in the present invention (e.g., bind TNF□ with high affinity and possess low immunogenicity). A functional equivalent of TNF receptor molecule also includes modified TNF receptor molecules that functionally resemble TNF receptor molecules that can be used in the present invention (e.g., bind TNF□ with high affinity and possess low immunogenicity). For example, a functional equivalent of TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1987-2000).

Cytokines include any known cytokine. See, e.g., CopewithCytokines.com. Cytokine antagonists include, but are not limited to, any antibody, fragment or mimetic, any soluble receptor, fragment or mimetic, any small molecule antagonist, or any combination thereof.

Therapeutic Treatments. Any method of the present invention can comprise a method for treating a Mut-IL18 or Mut-IL-18R mediated disorder or disease, comprising administering an effective amount of a composition or pharmaceutical composition comprising at least one Mut-IL18 or Mut-IL-18R antibody or protein to a cell, tissue, organ, animal or patient in need of such modulation, treatment or therapy. Such a method can optionally further comprise co-administration or combination therapy for treating such disorders or diseases, wherein the administering of said at least one Mut-IL18 or Mut-IL-18R antibody or protein, further comprises administering, before concurrently, and/or after, at least one selected from at least one at least one selected from at least one TNF antagonist (e.g., but not limited to a TNF antibody or fragment, a soluble TNF receptor or fragment, fusion proteins thereof, or a small molecule TNF antagonist), an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist.

Protein Dosing

Typically, treatment of pathologic conditions is effected by administering an effective amount or dosage of at least one Mut-IL8 or Mut-IL-18R protein composition that total, on average, a range from at least about 0.001 ng to 500 milligrams of at least one Mut-IL8 or Mut-IL-18R protein per kilogram of patient per dose, and preferably from at least about 0.1 ng to 100 milligrams antibody /kilogram of patient per single or multiple administration, depending upon the specific activity of contained in the composition. Alternatively, the effective serum concentration can comprise 0.0001 ng-0.05 mg/ml serum concentration per single or multiple adminstration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses of at least one protein can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 micrograms or milligrams/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5., 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 ng or μg/ml serum concentration per single or multiple administration, or any range, value or fraction thereof

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 μg to 100 milligrams per kilogram of body weight. Ordinarily 0.0001 to 50, and preferably 0.001 to 10 milligrams per kilogram per administration or in sustained release form is effective to obtain desired results.

As a non-limiting example, treatment of humans or animals can be provided as a one-time or periodic dosage of at least one antibody of the present invention 0.1 to 100 μg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90, 100, 200, 300, 400, 500, 600, 700, 800, 900, 1000, 2000 or 3000 μg/kg, per day, or 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or20 years, or any combination thereof, using single, infusion or repeated doses.

Dosage forms (composition) suitable for internal administration generally contain from about 0.00001 milligram to about 500 milligrams of active ingredient per unit or container. In these pharmaceutical compositions the active ingredient will ordinarily be present in an amount of about 0.5-99.999% by weight based on the total weight of the composition.

Typically, treatment of pathologic conditions is effected by administering an effective amount or dosage of at least one Mut-IL18 or Mut-IL-18R antibody composition that total, on average, a range from at least about 0.00001 to 500 milligrams of at least one Mut-IL18 or Mut-IL-18R antibody per kilogram of patient per dose, and preferably from at least about 0.0001 to 100 milligrams antibody /kilogram of patient per single or multiple administration, depending upon the specific activity of contained in the composition. Alternatively, the effective serum concentration can comprise 0.0001-500 μg/ml serum concentration per single or multiple adminstration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Antibody Dosing

Typically, treatment of pathologic conditions is effected by administering an effective amount or dosage of at least one Mut-L18 or Mut-IL-18R antibody composition that total, on average, a range from at least about 0.001 ng to 500 milligrams of at least one Mut-IL 18 or Mut-IL-18R antibody per kilogram of patient per dose, and preferably from at least about 0.1 ng to 100 milligrams antibody /kilogram of patient per single or multiple administration, depending upon the specific activity of contained in the composition. Alternatively, the effective serum concentration can comprise 0.0001 ng-0.05 mg/ml serum concentration per single or multiple adminstration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses of at least one antibody can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5., 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably 0.1 to 10 milligrams per kilogram per administration or in sustained release form is effective to obtain desired results.

As a non-limiting example, treatment of humans or animals can be provided as a one-time or periodic dosage of at least one antibody of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.1 milligram to about 500 milligrams of active ingredient per unit or container. In these pharmaceutical compositions the active ingredient will ordinarily be present in an amount of about 0.5-99.999% by weight based on the total weight of the composition.

Administration

For parenteral administration, the antibody or protein can be formulated as a solution, suspension, emulsion or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Alternative Administration

Many known and developed modes of can be used according to the present invention for administering pharmaceutically effective amounts of at least one Mut-IL18 or Mut-IL-18R antibody according to the present invention. While pulmonary administration is used in the following description, other modes of administration can be used according to the present invention with suitable results.

Mut-IL18 or Mut-IL-18R antibodies of the present invention can be delivered in a carrier, as a solution, emulsion, colloid, or suspension, or as a dry powder, using any of a variety of devices and methods suitable for administration by inhalation or other modes described here within or known in the art.

Parenteral Formulations and Administration

Formulations for parenteral administration can contain as common excipients sterile water or saline, polyalkylene glycols such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent such as aquous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

Alternative Delivery

The invention further relates to the administration of at least one Mut-IL18 or Mut-IL-18R antibody by parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal means. At least one Mut-IL18 or Mut-IL-18R antibody composition can be prepared for use for parenteral (subcutaneous, intramuscular or intravenous) or any other administration particularly in the form of liquid solutions or suspensions; for use in vaginal or rectal administration particularly in semisolid forms such as, but not limited to, creams and suppositories; for buccal, or sublingual administration such as, but not limited to, in the form of tablets or capsules; or intranasally such as, but not limited to, the form of powders, nasal drops or aerosols or certain agents; or transdermally such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement”; Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways such as electroporation, or to increase the mobility of charged drugs through the skin such as iontophoresis, or application of ultrasound such as sonophoresis (U.S. Pat. Nos. 4,309,989 and 4,767,402) (the above publications and patents being entirely incorporated herein by reference).

Pulmonary/Nasal Administration

For pulmonary administration, preferably at least one Mut-IL18 or Mut-IL-18R antibody composition is delivered in a particle size effective for reaching the lower airways of the lung or sinuses. According to the invention, at least one Mut-IL18 or Mut-IL-18R antibody can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of antibodies are also known in the art. All such devices can use of formulations suitable for the administration for the dispensing of antibody in an aerosol. Such aerosols can be comprised of either solutions (both aqueous and non aqueous) or solid particles. Metered dose inhalers like the Ventolin® metered dose inhaler, typically use a propellent gas and require actuation during inspiration (See, e.g., WO 94/16970, WO 98/35888). Dry powder inhalers like Turbuhaler™ (Astra), Rotahaler® (Glaxo), Diskus® (Glaxo), Spiros™ inhaler (Dura), devices marketed by Inhale Therapeutics, and the Spinhaler® powder inhaler (Fisons), use breath-actuation of a mixed powder (U.S. Pat. No. 4668218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent® nebulizer (Mallinckrodt), and the Acorn II® nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention. Preferably, a composition comprising at least one Mut-IL18 or Mut-IL-18R antibody is delivered by a dry powder inhaler or a sprayer. There are a several desirable features of an inhalation device for administering at least one antibody of the present invention. For example, delivery by the inhalation device is advantageously reliable, reproducible, and accurate. The inhalation device can optionally deliver small dry particles, e.g. less than about 10 μm, preferably about 1-5 μm, for good respirability.

Administration of Mut-IL18 or Mut-IL-18R Antibody Compositions as a Spray

A spray including Mut-IL18 or Mut-IL-18R antibody composition can be produced by forcing a suspension or solution of at least one Mut-IL18 or Mut-IL-18R antibody through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one Mut-IL18 or Mut-IL-18R antibody composition delivered by a sprayer have a particle size less than about 10 μm, preferably in the range of about 1 μm to about 5 μm, and most preferably about 2 μm to about 3 μm.

Formulations of at least one Mut-IL18 or Mut-IL-18R protein or antibody composition suitable for use with a sprayer typically include antibody or protein compositions in an aqueous solution at a concentration of about 0.0000001 mg to about 1000 mg of at least one Mut-IL18 or Mut-IL-18R antibody or protein composition per ml of solution or mg/gm, or any range or value therein, e.g., but not Imited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 ng or μg or mg/ml or ng or μg or mg/gm. The formulation can include agents such as an excipient, a buffer, an isotonicity agent, a preservative, a surfactant, and, preferably, zinc. The formulation can also include an excipient or agent for stabilization of the antibody composition, such as a buffer, a reducing agent, a bulk protein, or a carbohydrate. Bulk proteins useful in formulating antibody compositions include albumin, protamine, or the like. Typical carbohydrates useful in formulating antibody compositions include sucrose, mannitol, lactose, trehalose, glucose, or the like. The antibody composition formulation can also include a surfactant, which can reduce or prevent surface-induced aggregation of the antibody or protein composition caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of a protein such as Mut-IL18 or Mut-IL-18R antibodies, or specified portions or variants, can also be included in the formulation.

Administration of Mut-IL18 or Mut-IL-18R Antibody Compositions by a Nebulizer

antibody composition can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of antibody composition through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of antibody composition either directly or through a coupling fluid, creating an aerosol including the antibody composition. Advantageously, particles of antibody composition delivered by a nebulizer have a particle size less than about 10 μm, preferably in the range of about 1 μm to about 5 μm, and most preferably about 2 μm to about 3 μm.

Formulations of at least one Mut-IL18 or Mut-IL-18R antibody suitable for use with a nebulizer, either jet or ultrasonic, typically include a concentration of about 0.1 mg to about 100 mg of at least one Mut-IL18 or Mut-IL-18R antibody protein per ml of solution. The formulation can include agents such as an excipient, a buffer, an isotonicity agent, a preservative, a surfactant, and, preferably, zinc. The formulation can also include an excipient or agent for stabilization of the at least one Mut-IL18 or Mut-IL-18R antibody composition, such as a buffer, a reducing agent, a bulk protein, or a carbohydrate. Bulk proteins useful in formulating at least one Mut-IL18 or Mut-IL-18R antibody compositions include albumin, protamine, or the like. Typical carbohydrates useful in formulating at least one Mut-IL18 or Mut-IL-18R antibody include sucrose, mannitol, lactose, trehalose, glucose, or the like. The at least one Mut-IL18 or Mut-IL-18R antibody formulation can also include a surfactant, which can reduce or prevent surface-induced aggregation of the at least one Mut-IL18 or Mut-IL-18R antibody caused by atomization of the solution in forming an aerosol. Various conventional surfactants can be employed, such as polyoxyethylene fatty acid esters and alcohols, and polyoxyethylene sorbital fatty acid esters. Amounts will generally range between 0.001 and 4% by weight of the formulation. Especially preferred surfactants for purposes of this invention are polyoxyethylene sorbitan mono-oleate, polysorbate 80, polysorbate 20, or the like. Additional agents known in the art for formulation of a protein such as antibody protein can also be included in the formulation.

Administration of Mut-IL18 or Mut-IL-18R Antibody Compositions By A Metered Dose Inhaler

In a metered dose inhaler (MDI), a propellant, at least one Mut-IL18 or Mut-IL-18R antibody, and any excipients or other additives are contained in a canister as a mixture including a liquefied compressed gas. Actuation of the metering valve releases the mixture as an aerosol, preferably containing particles in the size range of less than about 10 μm, preferably about 1 μm to about 5 μm, and most preferably about 2 μm to about 3 μm. The desired aerosol particle size can be obtained by employing a formulation of antibody composition produced by various methods known to those of skill in the art, including jet-milling, spray drying, critical point condensation, or the like. Preferred metered dose inhalers include those manufactured by 3M or Glaxo and employing a hydrofluorocarbon propellant.

Formulations of at least one Mut-IL18 or Mut-IL-18R antibody for use with a metered-dose inhaler device will generally include a finely divided powder containing at least one Mut-IL18 or Mut-IL-18R antibody as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluroalkane-134a), HFA-227 (hydrofluroalkane-227), or the like. Preferably the propellant is a hydrofluorocarbon. The surfactant can be chosen to stabilize the at least one Mut-IL18 or Mut-IL-18R antibody as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol. Additional agents known in the art for formulation of a protein such as protein can also be included in the formulation.

One of ordinary skill in the art will recognize that the methods of the current invention can be achieved by pulmonary administration of at least one Mut-IL18 or Mut-IL-18R antibody compositions via devices not described herein.

Oral Formulations and Administration

Formulations for oral rely on the co-administration of adjuvants (e.g., resorcinols and nonionic surfactants such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant such as magnesium stearate, paraben, preserving agent such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds descnbed in U.S. Pat. No. 5,879,681 and U.S. Pat. No. 5,5,871,753 are used to deliver biologically active agents orally are known in the art.

Mucosal Formulations and Administration

For absorption through mucosal surfaces, compositions and methods of administering at least one Mut-IL18 or Mut-IL-18R antibody include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g. suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration excipients include sugars, calcium stearate, magnesium stearate, pregelinatined starch, and the like (U.S. Pat. No. 5,849,695).

Transdermal Formulations and Administration

For transdermal administration, the at least one Mut-IL18 or Mut-IL-18R antibody is encapsulated in a delivery device such as a liposome or polymeric nanoparticles, microparticle, microcapsule, or microspheres (referred to collectively as microparticles unless otherwise stated). A number of suitable devices are known, including microparticles made of synthetic polymers such as polyhydroxy acids such as polylactic acid, polyglycolic acid and copolymers thereof, polyorthoesters, polyanhydrides, and polyphosphazenes, and natural polymers such as collagen, polyamino acids, albumin and other proteins, alginate and other polysaccharides, and combinations thereof (U.S. Pat. No. 5,814,599).

Prolonged Administration and Formulations

It can be sometimes desirable to deliver the compounds of the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b) e.g. a zinc tannate salt. Additionally, the compounds of the present invention or, preferably, a relatively insoluble salt such as those just described, can be formulated in a gel, for example, an aluminum monostearate gel with, e.g. sesame oil, suitable for injection. Particularly preferred salts are zinc salts, zinc tannate salts, pamoate salts, and the like. Another type of slow release depot formulation for injection would contain the compound or salt dispersed for encapsulated in a slow degrading, non-toxic, non-antigenic polymer such as a polylactic acid/polyglycolic acid polymer for example as described in U.S. Pat. No. 3,773,919. The compounds or, preferably, relatively insoluble salts such as those described above can also be formulated in cholesterol matrix silastic pellets, particularly for use in animals. Additional slow release, depot or implant formulations, e.g. gas or liquid liposomes are known in the literature (U.S. Pat. Nos. 5,770,222 and “Sustained and Controlled Release Drug Delivery Systems”, J. R. Robinson ed., Marcel Dekker, Inc., N.Y., 1978).

Having generally described the invention, the same will be more readily understood by reference to the following examples, which are provided by way of illustration and are not intended as limiting.

### EXAMPLE 1

### Cloning and Expression of Mut-IL18 or Mut-IL-18R Protein or Antibody in Mammalian Cells

A typical mammalian expression vector contains at least one promoter element, which mediates the initiation of transcription of mRNA, the antibody coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES lneo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−(Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded protein or antibody, e.g., as a desired portion of at least one of SEQ ID NOS: 1-2. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 (1991); Bebbington, et al., Bio/Technology 10: 169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are used for the production of antibodies or proteins of the present invention.

The expression vectors pC1 and pC4 contain the strong promoter (LTR) of the Rous Sarcoma Virus (Cullen, et al., Molec. Cell. Biol. 5:438-447 (1985)) plus a fragment of the CMV-enhancer (Boshart, et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

Cloning and Expression in CHO Cells

The vector pC4 is used for the expression of Mut-IL18 or Mut-IL-18R antibody or protein, e.g., using a coding sequence for at least one of SEQ ID NOS: 1-2. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (e.g., alpha minus MEM, Life Technologies, Gaithersburg, Md.) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained that contain the amplified gene integrated into one or more chromosome(s) of the host cell.

Plasmid pC4 contains coding DNA for expressing the gene of interest under control of the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the Mut-IL18 or Mut-IL-18R in a regulated way in mammalian cells (M. Gossen, and H. Bujard, Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It can be advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the desired Mut-IL18 or Mut-IL-18R antibody or protein is used, e.g., DNA or RNA coding for at least one of SEQ ID NOS: 1-2, corresponding to at least one portion of at least one Mut-IL18 or Mut-IL-18R antibody protein of the present invention, according to known method steps.

The isolated encoding DNA and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained that grow at a concentration of 100-200 mM. Expression of the desired gene product is analyzed, for instance, by SDS-PAGE and Western blot or by reverse phase HPLC analysis.

### EXAMPLE 2

### Generation of Antibodies Reactive With Human Mut-IL18 or Mut-IL-18R Using Transgenic Mice

### Summary

Transgenic mice have been used that contain human heavy and light chain immunoglobulin genes to generate high affinity, completely human, monoclonal antibodies that can be used therapeutically to inhibit the action of Mut-IL18 or Mut-IL-18R for the treatment of one or more Mut-IL18 or Mut-IL-18R-mediated disease. (CBA/J x C57/BL6/J) F₂ hybrid mice containing human variable and constant region antibody transgenes for both heavy and light chains are immunized with human recombinant Mut-IL18 or Mut-IL-18R (Taylor et al., Intl. Immunol. 6:579-591 (1993); Lonberg, et al., Nature 368:856-859 (1994); Neuberger, M., Nature Biotech. 14:826 (1996); Fishwild, et al., Nature Biotechnology 14:845-851 (1996)). Several fusions yield one or more panels of completely human Mut-IL18 or Mut-IL-18R reactive IgG monoclonal antibodies. The completely human Mut-IL18 or Mut-IL-18R antibodies are further characterized. All are IgG1κ. Such antibodies are found to have affinity constants somewhere between 1×10⁹ and 9×10¹². The high affinities of these fully human monoclonal antibodies make them suitable candidates for therapeutic applications in Mut-IL18 or Mut-IL-18R related diseases, pathologies or disorders.

Abbreviations

BSA—bovine serum albumin

CO₂—carbon dioxide

DMSO—dimethyl sulfoxide

EIA—enzyme immunoassay

FBS—fetal bovine serum

H₂O₂—hydrogen peroxide

HRP—horseradish peroxidase\

ID—interadermal

Ig—immunoglobulin

Mut-IL18 or Mut-IL-18R—Interleukin-18/Interleukin receptor muteins

IP—intraperitoneal

IV—intravenous

Mab—monoclonal antibody

OD—optical density

OPD—o-Phenylenediamine dihydrochloride

PEG—polyethylene glycol

PSA—penicillin, streptomycin, amphotericin

RT—room temperature

SQ—subcutaneous

v/v—volume per volume

w/v—weight per volume

Materials and Methods

Animals

Transgenic mice that can express human antibodies are known in the art (and are commecially available (e.g., from GenPharm International, San Jose, Calif.; Abgenix, Freemont, Calif., and others) that express human immunoglobulins but not mouse IgM or Igκ. For example, such transgenic mice contain human sequence transgenes that undergo V(D)J joining, heavy-chain class switching, and somatic mutation to generate a repertoire of human sequence immunoglobulins (Lonberg, et al., Nature 368:856-859 (1994)). The light chain transgene can be derived, e.g., in part from a yeast artificial chromosome clone that includes nearly half of the germline human Vκ region. In addition, the heavy-chain transgene can encode both human μ and human γ1(Fishwild, et al., Nature Biotechnology 14:845-851 (1996)) and/or γ3 constant regions. Mice derived from appropriate genotopic lineages can be used in the immunization and fusion processes to generate fully human monoclonal antibodies to Mut-IL18 or Mut-IL-18R.

Immunization

One or more immunization schedules using at least one Mut-IL18 or Mut-IL-18R protein as an immunogen as generated according to know methods (e.g., as provided in Example 1) can be used to generate the Mut-IL18 or Mut-IL-18R human hybridomas. The first several fusions can be performed after the following exemplary immunization protocol, but other similar known protocols can be used. Several 14-20 week old female and/or surgically castrated transgenic male mice are immunized IP and/or ID with 1-1000 μg of recombinant human Mut-IL18 or Mut-IL-18R protein emulsified with an equal volume of TITERMAX or complete Freund's adjuvant in a final volume of 100-400 μL (e.g., 200). Each mouse can also optionally receive 1-10 μg in 100 μL physiological saline at each of 2 SQ sites. The mice can then be immunized 1-7, 5-12, 10-18, 17-25 and/or 21-34 days later IP (1-400 μg) and SQ (1-400 μg×2) with Mut-IL18 or Mut-IL-18R emulsified with an equal volume of TITERMAX or incomplete Freund's adjuvant. Mice can be bled 12-25 and 25-40 days later by retro-orbital puncture without coagulant. The blood is then allowed to clot at RT for one hour and the serum is collected and titered using an Mut-IL18 or Mut-IL-18R EIA assay according to known methods. Fusions are performed when repeated injections do not cause titers to increase. At that time, the mice can be given a final IV booster injection of 1-400 μg Mut-IL18 or Mut-IL-18R diluted in 100 μL physiological saline. Three days later, the mice can be euthanized by cervical dislocation and the spleens removed aseptically and immersed in 10 mL of cold phosphate buffered saline (PBS) containing 100 U/mL penicillin, 100 μg/mL streptomycin, and 0.25 μg/mL amphotericin B (PSA). The splenocytes are harvested by sterilely perfusing the spleen with PSA-PBS. The cells are washed once in cold PSA-PBS, counted using Trypan blue dye exclusion and resuspended in RPMI 1640 media containing 25 mM Hepes.

Cell Fusion

Fusion can be carried out at a 1:1 to 1:10 ratio of murine myeloma cells to viable spleen cells according to known methods, e.g., as known in the art. As a non-limiting example, spleen cells and myeloma cells can be pelleted together. The pellet can then be slowly resuspended, over 30 seconds, in 1 mL of 50% (w/v) PEG/PBS solution (PEG molecular weight 1,450, Sigma) at 37° C. The fusion can then be stopped by slowly adding 10.5 mL of RPMI 1640 medium containing 25 mM Hepes (37° C.) over 1 minute. The fused cells are centrifuged for 5 minutes at 500-1500 rpm. The cells are then resuspended in HAT medium (RPMI 1640 medium containing 25 mM Hepes, 10% Fetal Clone I serum (Hyclone), 1 mM sodium pyruvate, 4 mM L-glutamine, 10 μg/mL gentamicin, 2.5% Origen culturing supplement (Fisher), 10% 653-conditioned RPMI 1640/Hepes media, 50 μM 2-mercaptoethanol, 100 μM hypoxanthine, 0.4 μM aminopterin, and 16 μM thymidine) and then plated at 200 μL/well in fifteen 96-well flat bottom tissue culture plates. The plates are then placed in a humidified 37° C. incubator containing 5% CO₂ and 95% air for 7-10 days.

Detection of Human IgG Mut-IL18 or Mut-IL-18R Antibodies in Mouse Serum

Solid phase EIA's can be used to screen mouse sera for human IgG antibodies specific for human Mut-IL18 or Mut-IL-18R protein. Briefly, plates can be coated with Mut-IL18 or Mut-IL-18R protein at 2 μg/mL in PBS overnight. After washing in 0.15M saline containing 0.02% (v/v) Tween 20, the wells can be blocked with 1% (w/v) BSA in PBS, 200 μL/well for 1 hour at RT. Plates are used immediately or frozen at −20° C. for future use. Mouse serum dilutions are incubated on the Mut-IL18 or Mut-IL-18R coated plates at 50 μL/well at RT for 1 hour. The plates are washed and then probed with 50 μL/well HRP-labeled goat human IgG, Fc specific diluted 1:30,000 in 1% BSA-PBS for 1 hour at RT. The plates can again be washed and 100 μL/well of the citrate-phosphate substrate solution (0.1M citric acid and 0.2M sodium phosphate, 0.01% H₂O₂ and 1 mg/mL OPD) is added for 15 minutes at RT. Stop solution (4N sulfuric acid) is then added at 25 μL/well and the OD's are read at 490 nm via an automated plate spectrophotometer.

Detection of Completely Human Immunoglobulins in Hybridoma Supernates

Growth positive hybridomas secreting fully human immunoglobulins can be detected using a suitable EIA. Briefly, 96 well pop-out plates (VWR, 610744) can be coated with 10 μg/mL goat human IgG Fc in sodium carbonate buffer overnight at 4° C. The plates are washed and blocked with 1% BSA-PBS for one hour at 37° C. and used immediately or frozen at −20° C. Undiluted hybridoma supernatants are incubated on the plates for one hour at 37° C. The plates are washed and probed with HRP labeled goat human kappa diluted 1:10,000 in 1% BSA-PBS for one hour at 37° C. The plates are then incubated with substrate solution as described above.

Determination of Fully Human Mut-IL18 or Mut-IL-18R Reactivity

Hybridomas, as above, can be simultaneously assayed for reactivity to Mut-IL18 or Mut-IL-18R using a suitable RIA or other assay. For example, supernatants are incubated on goat human IgG Fc plates as above, washed and then probed with radiolabled Mut-IL18 or Mut-IL-18R with appropriate counts per well for 1 hour at RT. The wells are washed twice with PBS and bound radiolabled Mut-IL18 or Mut-IL-18R is quantitated using a suitable counter.

Human IgG1κ Mut-IL18 or Mut-IL-18R secreting hybridomas can be expanded in cell culture and serially subcloned by limiting dilution. The resulting clonal populations can be expanded and cryopreserved in freezing medium (95% FBS, 5% DMSO) and stored in liquid nitrogen.

Isotyping

Isotype determination of the antibodies can be accomplished using an EIA in a format similar to that used to screen the mouse immune sera for specific titers. Mut-IL18 or Mut-IL-18R protein can be coated on 96-well plates as described above and purified antibody at 2 μg/mL can be incubated on the plate for one hour at RT. The plate is washed and probed with HRP labeled goat human IgG₁ or HRP labeled goat human IgG₃ diluted at 1:4000 in 1% BSA-PBS for one hour at RT. The plate is again washed and incubated with substrate solution as described above.

Binding Kinetics of Human Human Mut-IL18 or Mut-IL-18R Antibodies With Human Mut-IL18 or Mut-IL-18R

Binding characteristics for antibodies can be suitably assessed using an Mut-IL18 or Mut-IL-18R capture EIA and BIAcore technology, for example. Graded concentrations of purified human Mut-IL18 or Mut-IL-18R antibodies can be assessed for binding to EIA plates coated with 2 μg/mL of Mut-IL18 or Mut-IL-18R in assays as described above. The OD's can be then presented as semi-log plots showing relative binding efficiencies.

Quantitative binding constants can be obtained, e.g., as follows, or by any other known suitable method. A BIAcore CM-5 (carboxymethyl) chip is placed in a BIAcore 2000 unit. HBS buffer (0.01 M HEPES, 0.15 M NaCl, 3 mM EDTA, 0.005% v/v P20 surfactant, pH 7.4) is flowed over a flow cell of the chip at 5 μL/minute until a stable baseline is obtained. A solution (100 μL) of 15 mg of EDC (N-ethyl-N′-(3-dimethyl-aminopropyl)-carbodiimide hydrochloride) in 200 μL water is added to 100 μL of a solution of 2.3 mg of NHS (N-hydroxysuccinimide) in 200 μL water. Forty (40) μL of the resulting solution is injected onto the chip. Six μL of a solution of human Mut-IL18 or Mut-IL-18R (15 μg/mL in 10 mM sodium acetate, pH 4.8) is injected onto the chip, resulting in an increase of ca. 500 RU. The buffer is changed to TBS/Ca/Mg/BSA running buffer (20 mM Tris, 0.15 M sodium chloride, 2 mM calcium chloride, 2 mM magnesium acetate, 0.5% Triton X-100, 25 μg/mL BSA, pH 7.4) and flowed over the chip overnight to equilibrate it and to hydrolyze or cap any unreacted succinimide esters.

Antibodies are dissolved in the running buffer at 33.33, 16.67, 8.33, and 4.17 nM. The flow rate is adjusted to 30 μL/min and the instrument temperature to 25° C. Two flow cells are used for the kinetic runs, one on which Mut-IL18 or Mut-IL-18R protein had been immobilized (sample) and a second, underivatized flow cell (blank). 120 μL of each antibody concentration is injected over the flow cells at 30 μL/min (association phase) followed by an uninterrupted 360 seconds of buffer flow (dissociation phase). The surface of the chip is regenerated (Interleukin-18/Interleukin receptor muteins /antibody complex dissociated) by two sequential injections of 30 μL each of 2 M guanidine thiocyanate.

Analysis of the data is done using BIA evaluation 3.0 or CLAMP 2.0, as known in the art. For each antibody concentration the blank sensogram is subtracted from the sample sensogram. A global fit is done for both dissociation (k_{d}, sec⁻¹) and association (kₐ, mol⁻¹ sec⁻¹) and the dissociation constant (K_{D}, mol) calculated (k_{d}/kₐ). Where the antibody affinity is high enough that the RUs of antibody captured are >100, additional dilutions of the antibody are run.

Results and Discussion

Generation of Human Mut-IL18 or Mut-IL-18R Monoclonal Antibodies

Several fusions are performed and each fusion is seeded in 15 plates (1440 wells/fusion) that yield several dozen antibodies specific for human Mut-L 18 or Mut-IL-18R protein. Of these, some are found to consist of a combination of human and mouse Ig chains. The remaining hybridomas secret Mut-IL18 or Mut-IL-18R antibodies consisting solely of human heavy and light chains. Of the human hybridomas all are expected to be IgG1κ.

Binding Kinetics of Human Human Mut-IL18 or Mut-IL-18R Antibodies

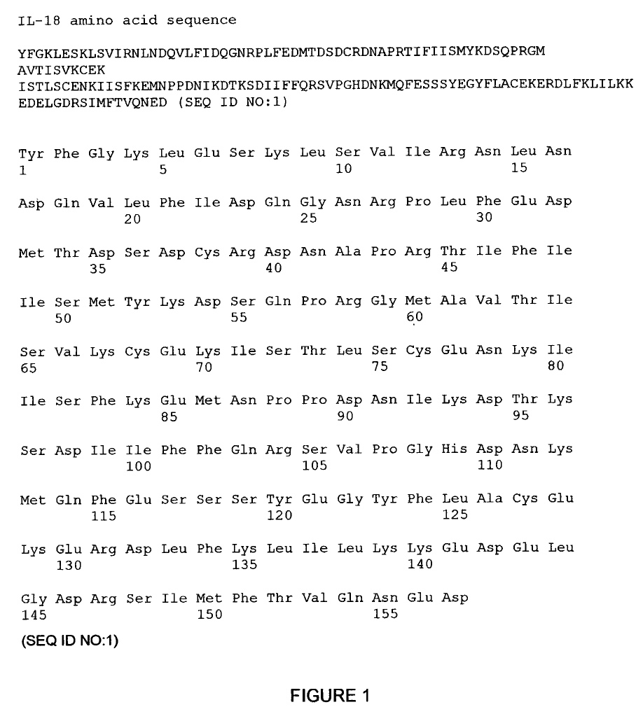

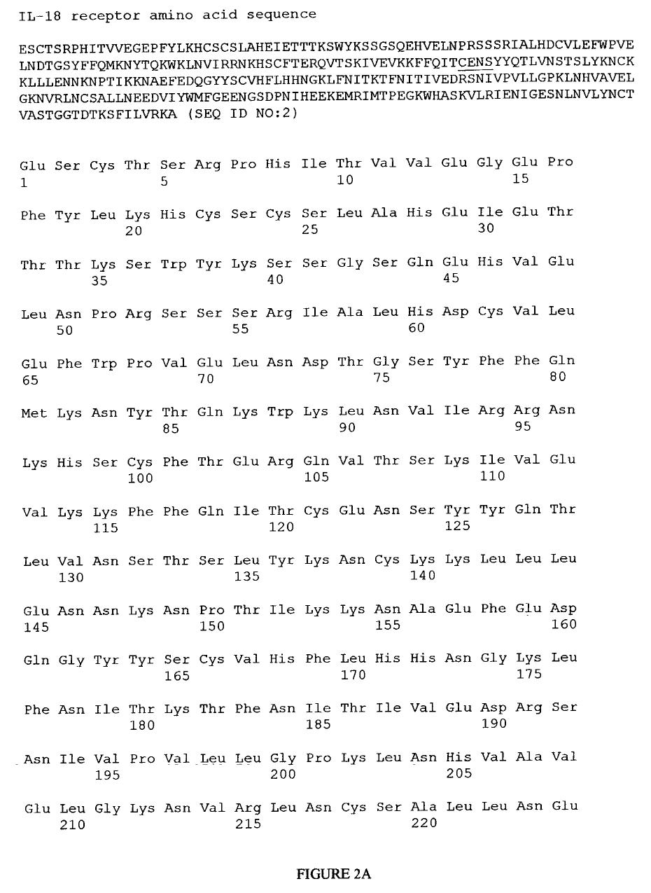

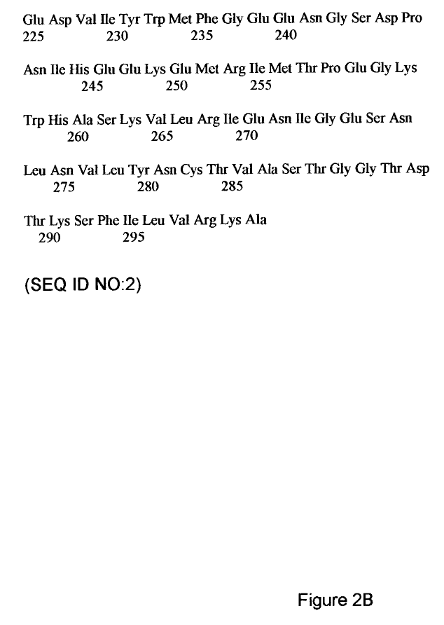

ELISA analysis confirms that purified antibody from most or all of these hybridomas bind Mut-IL18 or Mut-IL-18R protein in a concentration-dependent manner. FIGS. 1-2 show the results of the relative binding efficiency of these antibodies. In this case, the avidity of the antibody for its cognate antigen (epitope) is measured. It should be noted that binding Mut-IL18 or Mut-IL-18R directly to the EIA plate can cause denaturation of the protein and the apparent binding affinities cannot be reflective of binding to undenatured protein. Fifty percent binding is found over a range of concentrations.

Quantitative binding constants are obtained using BIAcore analysis of the human antibodies and reveals that several of the human monoclonal antibodies are very high affinity with KDin the range of 1×10⁻⁸ to 7×10⁻¹².

Conclusions

Several fusions are performed utilizing splenocytes from hybrid mice containing human variable and constant region antibody transgenes that are immunized with human Mut-IL18 or Mut-IL-18R. A set of several completely human Mut-IL18 or Mut-IL-18R reactive IgG monoclonal antibodies of the IgGlK isotype are generated. The completely human Mut-IL18 or Mut-IL-18R antibodies are further charactenzed. Several of generated antibodies have affinity constants between 1×10⁸ and 9×10¹². The unexpectedly high affinities of these fully human monoclonal antibodies make them suitable for therapeutic applications in Mut-IL18 or Mut-IL-18R-dependent diseases, pathologies or related conditions.

### EXAMPLE 3

### Generation of IL-18 Muteins

IL-1 and IL-1 receptor are structurally homologous to IL-18 and IL-18 respectively. Using the crystal structure of IL-1β with its receptor from the Brookhaven Data Bank, a model of IL-18/IL-18receptor was constructed. Amino acids were electronically mutated, from IL-1β and of IL-1β receptor to the corresponding amino acids in human IL-18 and IL-18 receptor. Additions and deletions were handled by performing loop searches anchored at residues appearing on both molecules. Loops were examined for bond angles, interaction of backbone and side chains and rationality of position. The resulting structure was subjected to minimization and dynamics. Individual amino acids in IL-18 were examined and their interaction with the IL-18 receptor evaluated. Based on the model, rational substitutions were suggested that would either retain or alter IL-18 activity. The substitutions defined here are not meant to be the only substitutions possible or to limit the utility of this model. The muteins identified using this model are useful as IL-18 agonists, IL-18 antagonists, for raising anti-IL-18 antibodies and for substitution for IL-18 in assays, models, and other IL-18 functions.

Using the crystal structure of IL-1 with its receptor, the sequence of IL-1 was aligned with IL18.

| | |
|---|---|
| | 1 |
| IL-1 | --APVRSLNC TLRDSQQKSL VM---SGPYE LKALHLQGQD MEQQVVFSMS |
| IL-18 | YFGKLESKLS VIRNLNDQVL FIDQGNRPLF EDMTDSDCRD NAPRTIFIIS |
| | 51 |
| IL-1 | FVQGEESNDK IPVALGLKEK NLYLSCVLKD DKPTLQLESV DPKNYPKKKM |
| IL-18 | MYKDSQPRGM AVTISVKCEK ISTLSC---- ENKIISFKEM NPPDNIKDTK |
| | 101 |
| IL-1 | EKRFVFNKI- -EINNKLEFE SAQFPNWYIS TSQAENMPVF LGGTKGGQDI |
| IL-18 | SDIIFFQRSV PGHDNKMQFE SSSYEGYFLA CEKERDLFKL I--LKKEDEL |
| | 151 |
| IL-1 | TDFTMQFVSS --- |
| IL-18 | GDRSIMFTVQ NED |

Initial amino acid numbering refers to the positions in IL-1 and the IL-1 receptor. Once the IL-18/L-18 receptor structure was complete, the structure was renumbered to be consistent with IL-18/IL-18 receptor numbering. The amino acids in IL-1 were electronically mutated to the IL-18 sequence. Additions or deletions were ignored at this point.

The sequence of the IL-1 receptor was aligned with the sequence for the IL-18 receptor.

| | |
|---|---|
| | 1 |
| IL-1r | --CKEREEKI ILVSSANEID VRPCPLNPNE HKGTITWYK- --DDSKTPVS |
| IL-18r | ESCTSRPHIT VVEGEPFYLK HCSCSLAHEI ETTTKSWYKS SGSQEHVELN |
| | 51 |
| IL-1r | TEQASRIHQH KEKLWFVPAK VEDSGHYYCV VRNSSYCLRI KISAKFVENE |
| IL-18r | PRSSSRIALH DCVLEFWPVE LNDTGSYFFQ MKN--YTQKW KLN-- |
| VIRRN | |
| | 101 |
| IL-1r | PNLCYNAQAI FKQKLPVAGD GGLVC--PYM EFFKNENNEL PKLQWYKDCK |
| I-18r | KHSCFTERQV TSKIVEVKKF FQITCENSYY QTLVNSTS-- ----LYKNCK |
| | 151 |
| IL-1r | PLLLDNIHFS GVKDRLIVMN VAEKHRGNYT CHASYTYLGK QYPITRVIEF |
| IL-18r | KLLLEN---- -NKNPTIKKN AEFEDQGYYS CVHFLHHNGK LFNITKTFNI |
| | 201 |
| IL-1r | ITLEENKPTR PVIVSPANET MEVDLGSQIQ LICNVTGQLS DIAYWKWNGS |
| IL-18r | TIVEDRSNIV PVLLGPKLNH VAVELGKNVR LNCSALLNEE DVIYWMFGEE |
| | 251 |
| IL-1r | VIDEDDPVLG EDY-YSVENP ANKRRSTLIT VLNISEIESR FYKHPFTCFA |
| IL-18r | --NGSDPNIH EEKEMRIMTP EGKWHAS--K VLRIENIGES NLNVLYNCTV |
| | 301 |
| IL-1r | KNTHGIDAAY IQLIYPVT |
| IL-18r | ASTGGTDTKS FILVRKA- |

Individual amino acids in the IL-1 receptor were electronically mutated and the potential effect of the changes on the interaction with IL-18 was evaluated. An examination of the structure amino acid-by-amino acid led to the following observations:

Cys¹ forms a disulfide bond with Cys⁸². In the IL-18 receptor the equivalent of Cys⁸² is absent.

The change of Val¹¹ to Glu creates possible hydrogen bonding with Arg³⁴ in IL-18, an amino acid different from that in IL-1. Arg²⁰ to Cys gives an apparently unpaired cysteine but Glu⁵⁷ becomes a cysteine as well and is in the immediate vicinity. This becomes a new disulfide bond but geometry needs to be adjusted. Pro²⁶ to His adds an aromatic residue that will interact with the two new aromatics in IL-18, Phe²⁵ and Phe¹³¹. The loop from 45-51 needs to be redone because of a bad bend resulting from the new proline at 46. A disulfide is created between 20 and 57. This is a very long bond (14.257 angstroms) and some geometric correction is necessary. Cys⁷⁴ to Phe removes the disulfide bond with 22. Cys⁸² to Thr removes the disulfide bond with Cys¹. Pro¹¹¹ to Glu gives hydrogen bonding potential with IL-18 Arg¹¹ and Lys¹⁰⁹ (both unchanged from IL-1). Gln¹⁰⁸ to Lys gives hydrogen bonding potential with the Gln¹⁵ to Asp IL-18 mutation. Asn¹⁹⁹ to Arg creates the possibility of π-π interactions with Phe¹⁵⁰ in IL-18. Tyr²⁵⁶ to Lys and Ser²⁵⁸ to Arg give possible hydrogen bonding with IL-18 Glu⁴ that was an Arg. The regions where additions and/or deletions in the two sequences were present were identified. There are 3 regions where the additions are involved in contact between IL-18 and the receptor. These are underlined on the alignment sequences above.

Insertions and Deletions From the Sequences

The sequence VLKD in IL-1 is an external loop with no receptor contact. This sequence is deleted in IL-18. A loop search was done using Cys⁷¹ and Ile⁸⁰ as anchor points and searching for ENKI. This deleted the four amino acids and created a new loop. Of the loops identified, 1QBA:Arg825 gave a good fit and positioned the side chains such that the Glu hydrogen binds with the side chain of Lys⁸³ and Tyr¹¹⁷ and Tyr¹²⁰ can form a π-π interaction. To remove the Gly¹³⁵⁻¹³⁶ in IL-1, a loop search was done, anchoring at Ile¹³⁴ and Gly¹⁴⁴ and searching for the loop LKKEDE. The loop 1AHJ:D/Glu134 that placed all hydrophilic residues on the surface was inserted. The sequence NED was added to the C-terminus in a trans configuration. This allowed hydrogen bonding with the Glu and Arg²⁵⁸ of the receptor. There were two deletions in the IL-18 receptor close together (SS and AK). Both of these were done together since they are part of a long beta structure. The loop was anchored at Met⁷⁶ and Val⁹¹ and the sequence KNYTQKWKLN was searched. There is only one loop that gives trans amide bonds, the tyrosine giving π-π interactions with Arg² and the Trp giving π-π interactions with His⁶, 1CHM:B/Met253. This was inserted, the side chains relaxed. To remove IHFSG from the IL-1 receptor, a loop search anchoring at Leu¹⁴⁵ and Ile¹⁶⁰ and searching for LLENNKNKPT was done. The loop 1LPB:B/Phe72 was inserted. To remove ELPKLQ from the IL-1 receptor, a loop search using Tyr¹²³ and Leu¹³⁸ as anchors was done, searching for QTLVNSTS. The loop 1FEC:B/Tyr182 was inserted. The sequence EGKWHAS—in IL-18 receptor was changed to—EGKWHAS and a loop search was done to modify the hairpin turn by removing EG. The anchor residues were Thr²⁶¹ and Lys²⁶⁷ and the search was for PEG. The loop 2FB4:H/Ser135 was inserted. To remove VI from the IL-1 receptor, a search for FGEEN was done, anchoring at Met²³⁹ and Gly²⁴⁷. The loop 1BRB:E/Arg67 was inserted. This region contacts the IL-18.

EEKEMRI needed the underlined E added. This is an IL-18 contact residue. This was an opportunity to remove some of the interactions between Trp²⁶⁸ and Ile²⁵⁹. A loop search was done using Thr²⁶¹ and Glu²⁵⁴ as anchor points and searching the sequence KEMRIM. The loop 1IND:L/Trp98 was inserted. A search for the sequence SSGSQE was done, anchoring at Lys³⁷ and His⁴¹. The loop 1SLT:B/Asn61 was inserted. The introduction of the VP into IL-18 was not simple. Based on the alignment, this is on the side of a loop, the tip of which contacts the receptor. The Asp probably hydrogen bonds with Lys¹¹⁴ of the receptor. A loop search, anchoring at Arg¹⁰³ and Asn¹⁰⁸ and searching the sequence SVPGHD, was done. The loop 1TDT:B/Thr212 was inserted.

For DQG in IL-18, a search for IDQGNRP, anchoring at Phe¹⁹ and Leu²⁴, was done. The loop 1PYS:B/Leu730 was inserted. The disulfide pairing in the IL-18 receptor was adjusted. From examination of the model, it was highly unlikely that cysteines 20 and 22 pair up. The most likely pairing was 22 with 57 and 20 with 1. This necessitated repositioning the loop of 73 to 82 to allow bringing the chain from 1 to 7 close enough to form the disulfide bond between 1 and 20. The loop 72-83 was deleted and the 1-7 sequence repositioned for disulfide bond formation by manipulating bond angles.

The torsional angle between Arg⁴ and Pro⁵ was modified from 149 degrees to 209 degrees. This placed the cysteine sulfurs 7.2 angstroms apart but with nothing in between. The distance between the sulfurs in cysteines 20 and 57 was 12 angstroms but the side chain on 20 was pointed in the wrong direction. Amino acids 1-4 of the receptor were manually positioned them so that they filled the gap around the Cys²⁰ and had the two cysteines close enough to form the disulfide bond. This was merged with the structure. Amino acids 1-4 were deleted from the receptor and a bond was formed between the new 4 and old 5. A loop search was then done using Thr² and Val¹⁰ as anchors and searching for SRPHITF. The loop from 1EZM:Phe54 was inserted. The loop between 72 and 83 was replaced. Using anchors at Asp⁶⁸ and Leu⁸⁵, a search for TGSYFFQMKNYTQKWK was done. The loop from 2CAS:Gly412 was inserted.

The resulting structure was refined as follows: The structure was minimize using steepest descent, 100 cycles, 8 angstroms for non-bonded cutoff, 100 dielectric, Tripos force field, kollman-all charges. A dynamics run was done (100 fs, random, NPT, 300 deg, 5 atm) followed by minimization (steepest descent, 100 cycles, 8 angstroms for non-bonded cutoff, 100 dielectric, Tripos force field, kollman-all charges). A final minimization was done (conjugate gradient, 100 cycles, 8 angstroms for non-bonded cutoff, 100 dielectric, Tripos force field, kollman-all charges). The resulting structure had inverted the chirality of Tyr¹. Tyr¹-Phe² was repositioned and local minimization done (conjugate gradient, 100 cycles, 8 angstroms for non-bonded cutoff, 100 dielectric, Tripos force field, kollman-all charges). The resulting model was examined amino acid-by-amino acid to determine the effect of potential amino acid substitutions on IL-18/IL-18 receptor interactions. The following observations were made: Tyr¹-Phe² These residues probably interact with the receptor and changing them would affect binding. Interaction is peripheral (at the edge of the receptor-ligand interface). I believe these residues to be important. Substitution by non-aromatic residues could reduce affinity. Lys⁴ may interact with Glu²⁴¹ and is peripheral. Leu⁵ is internal and could be substituted by valine. Glu⁶ probably interacts with Arg²⁴⁵. Lys⁸ interacts with the receptor and is critical. Ser¹⁰ could be replaced by Thr. Val¹¹ could be replaced by Ile. Ile¹² could be replaced by Val. Arg¹³ is probably a receptor contact residue. Leu¹⁵ may interact peripherally. Asp¹⁷ is a receptor contact residue and could be replaced by Asn. Gln¹⁸ may be a receptor contact residue. Leu²⁰ could be replaced by Val or Ile. Phe²¹ could be replaced by Tyr. Ile²² could be replaced by Val. Arg²⁷ is a peripheral receptor contact residue Leu²⁹ could be replaced by Val. Phe³⁰ is a residue contact residue that could be replaced by Tyr. Asp³⁵ is a receptor contact residue. DCRD (37-40) are receptor contact residues. Arg³⁹ is a receptor contact residue. Long shot, but it may be able to be substituted with a Trp. Ala⁴² is involved in a beta turn with Pro⁴³. Ala⁴² could be substituted with a Ser. Thr⁴⁵ could be replaced with Ser. Ile⁴⁶ could be replaced with Val. Phe⁴⁷ could be replaced with Tyr and it would add hydrogen bonding to Lys¹³⁵. Ser⁵⁰ could be replaced by Arg or Asn. Met⁵¹ is a possible receptor contact residue. Tyr⁵² could be replaced with Phe. Lys⁵³ is a critical receptor contact residue. Gln⁵⁶ is a receptor contact residue. A possible substitution would be Glu. Arg⁵⁸ is a receptor contact residue. Val⁶² is a receptor contact residue. Thr⁶³ could be replaced by Ala. Ile⁶⁴/Val⁶⁶ could be simultaneously replaced with Val⁶⁴/Ile⁶⁶. Glu⁶⁹ could be replaced with Gln, Asp or Asn. Ser⁷² could be replaced with Thr. Glu⁷⁷ could be replaced with Asp or Gln. Lys⁷⁹ could be replaced by Arg. Ser⁸² could be replaced with Thr. Glu⁸⁵ could be replaced with Asp. Met⁸⁶ could be replaced by Val, Gln or Asn. Asn⁸⁷ could be replaced with Gln. Pro⁸⁸ could be replaced with Ser. Ile⁹² could be replaced with Val. Asp⁹⁴ and Thr⁹⁵ are receptor contact residues. Asp⁹⁸ could be replaced with Glu or Asn. Phe¹⁰¹ could be replaced with Tyr. Arg¹⁰⁴ is receptor binding and critical. GHDN (108-111) are possible receptor contact residues. Gln¹¹⁴ could be replaced by Asn. Ser¹¹⁸ could be replaced by Thr. Tyr¹²⁰ could be replaced by Phe. Glu¹²¹ could be replaced by Asp. Tyr¹²³ could be replaced by Phe. Phe¹²⁴ could be replaced by Tyr. Ala¹²⁶ could be replaced by Thr. Lys¹²⁹ is a receptor contact residue. Glu¹³⁰ is a possible receptor contact residue. Arg¹³¹ is a receptor contact residue and critical. Asp¹³² is a receptor contact residue and critical. Leu¹³³ and Phe¹³⁴ are receptor contact residues and critical. Phe could be replaced by Tyr. Glu¹⁴¹ could be replaced by Lys or Asp. Ser¹⁴⁸ is a possible receptor contact residue. Simultaneous substitution of Asp¹¹⁰ by Arg and Ser¹⁴⁸ by Phe could increase binding of IL-18 to its receptor. Met¹⁵⁰ is a receptor contact residue. Phe¹⁵¹ is receptor contact and critical. Gln¹⁵⁴ could be replaced by Asn. Asn¹⁵⁵ could be replaced by Glu or Ser. Glu¹⁵⁶ could be replaced by Asp or Gln. Asp¹⁵⁷ could be replaced by Glu or Asn. A table was prepared in which the side chain and total amino acid surface exposure was calculated.

Residues that could be substituted were identified. Receptor binding residues were identified and a judgement was made as to whether they were on the periphery of the interface between IL-18 and the receptor. These would presumably be less sensitive to substitution. To create agonists, non-receptor contact amino acids could be substituted. To create antagonists, receptor contact residues could be substituted. To create an antigen for raising antibodies, non-surface exposed amino acids could be substituted. To create an antigen for raising neutralizing antibodies, receptor contact residues should be kept intact and both surface and non-surface exposed amino acids could be substituted. To avoid immunogenicity issues, surface amino acid substitutions should be avoided.

| | | | | | | | | | | | Substitutions | |
|---|---|---|---|---|---|---|---|---|---|---|---|---|
| | | Side | Total | | | | | | | | that would | |
| | AA | Chain | AA | Receptor | Possible | Buried | | | | | alter receptor | Peripheral |
| AA # | Type | exposed | exposed | Contact | substitutions | 25% | 20% | 15% | 10% | 5% | binding | residues |
| 1 | Tyr | 0.98 | 1.14 | X | | | | | | | | X |
| 2 | Phe | 0.99 | 1.01 | X | | | | | | | | X |
| 3 | Gly | — | 0.72 | | | | | | | | | |
| 4 | Lys | 0.99 | 0.89 | X | | | | | | | Glu | X |
| 5 | Leu | 0.16 | 0.26 | | Val | | X | | | | | |
| 6 | Glu | 1.20 | 0.89 | X | | | | | | | Ile | |
| 7 | Ser | 0.57 | 0.54 | | | | | | | | | |
| 8 | Lys | 0.67 | 0.50 | X | | | | | | | Asp | |
| 9 | Leu | 0.21 | 0.16 | | | | | | | | | |
| 10 | Ser | 0.00 | 0.00 | | Thr | | | | | X | | |
| 11 | Val | 0.08 | 0.07 | | Ile | | | | X | | | |
| 12 | Ile | 0.05 | 0.04 | | Val | | | | | X | | |
| 13 | Arg | 0.29 | 0.25 | X | | | | | | | Ile | X |
| 14 | Asn | 0.09 | 0.08 | | | | | | | | | |
| 15 | Leu | 0.79 | 0.65 | X | | | | | | | Arg | X |
| 16 | Asn | 0.31 | 0.37 | | | | | | | | | |
| 17 | Asp | 0.92 | 0.70 | X | Asn | | | | | | Lys | |
| 18 | Gln | 0.02 | 0.02 | X | | | | | | | | |
| 19 | Val | 0.26 | 0.18 | | | | | | | | | |
| 20 | Leu | 0.11 | 0.09 | | Val, Ile | | | X | | | | |
| 21 | Phe | 0.14 | 0.11 | | Tyr | | | X | | | | |
| 22 | Ile | 0.23 | 0.21 | | Val | X | | | | | | |
| 23 | Asp | 0.48 | 0.43 | | | | | | | | | |
| 24 | Gln | 1.10 | 1.04 | | | | | | | | | |
| 25 | Gly | — | 0.92 | | | | | | | | | |
| 26 | Asn | 0.40 | 0.37 | | | | | | | | | |
| 27 | Arg | 0.98 | 0.83 | X | | | | | | | Lys | X |
| 28 | Pro | 0.84 | 0.66 | | | | | | | | | |
| 29 | Leu | 0.56 | 0.47 | | Val | | | | | | | |
| 30 | Phe | 0.56 | 0.45 | X | Tyr | | | | | | Ala | X |
| 31 | Glu | 0.01 | 0.03 | | | | | | | | | |
| 32 | Asp | 0.24 | 0.15 | | | | | | | | | |
| 33 | Met | 0.03 | 0.03 | | | | | | | | | |
| 34 | Thr | 0.30 | 0.22 | | | | | | | | | |
| 35 | Asp | 0.77 | 0.69 | X | | | | | | | Lys | |
| 36 | Ser | 0.23 | 0.43 | | | | | | | | | |
| 37 | Asp | 1.05 | 0.76 | X | | | | | | | Phe | |
| 38 | Cys | 0.95 | 0.79 | X | | | | | | | Glu | X |
| 39 | Arg | 0.93 | 0.87 | X | Trp | | | | | | Ala | X |
| 40 | Asp | 1.00 | 0.66 | X | | | | | | | Trp | X |
| 41 | Asn | 0.66 | 0.47 | | | | | | | | | |
| 42 | Ala | 0.89 | 0.79 | | Ser | | | | | | | |
| 43 | Pro | 0.59 | 0.47 | | | | | | | | | |
| 44 | Arg | 0.40 | 0.37 | | | | | | | | | |
| 45 | Thr | 0.02 | 0.04 | | Ser | | | | | X | | |
| 46 | Ile | 0.54 | 0.45 | | Val | | | | | | | |
| 47 | Phe | 0.02 | 0.02 | | Tyr | | | | | X | | |
| 48 | Ile | 0.24 | 0.19 | | | | | | | | | |
| 49 | Ile | 0.05 | 0.07 | | | | | | | | | |
| 50 | Ser | 0.06 | 0.04 | | Arg, Asn | | | | X | | | |
| 51 | Met | 0.40 | 0.33 | X | | | | | | | Glu | |
| 52 | Tyr | 0.01 | 0.01 | | Phe | | | | | X | | |
| 53 | Lys | 0.77 | 0.61 | X | | | | | | | Gly | |
| 54 | Asp | 0.71 | 0.73 | | | | | | | | | |
| 55 | Ser | 0.87 | 0.77 | | | | | | | | | |
| 56 | Gln | 0.44 | 0.51 | X | Glu | | | | | | Ile | |
| 57 | Pro | 0.54 | 0.57 | | | | | | | | | |
| 58 | Arg | 0.98 | 0.92 | X | | | | | | | Ala | X |
| 59 | Gly | — | 0.65 | | | | | | | | | |
| 60 | Met | 0.38 | 0.33 | | | | | | | | | |
| 61 | Ala | 0.56 | 0.31 | | | | | | | | | |
| 62 | Val | 0.27 | 0.19 | X | | | | | | | Lys | |
| 63 | Thr | 0.00 | 0.00 | | Ala | | | | | X | | |
| 64 | Ile | 0.06 | 0.05 | | Val | | | | X | | | |
| 65 | Ser | 0.07 | 0.06 | | | | | | | | | |
| 66 | Val | 0.27 | 0.19 | | Ile | | X | | | | | |
| 67 | Lys | 0.08 | 0.06 | | | | | | | | | |
| 68 | Cys | 0.37 | 0.37 | | | | | | | | | |
| 69 | Glu | 0.95 | 0.87 | | Gln, Asp, | | | | | | | |
| | | | | | Asn | | | | | | | |
| 70 | Lys | 0.62 | 0.60 | | | | | | | | | |
| 71 | Ile | 0.48 | 0.41 | | | | | | | | | |
| 72 | Ser | 0.37 | 0.24 | | Thr | X | | | | | | |
| 73 | Thr | 0.12 | 0.09 | | | | | | | | | |
| 74 | Leu | 0.06 | 0.05 | | | | | | | | | |
| 75 | Ser | 0.00 | 0.00 | | | | | | | | | |
| 76 | Cys | 0.04 | 0.05 | | | | | | | | | |
| 77 | Glu | 0.42 | 0.32 | | Asp, Gln | | | | | | | |
| 78 | Asn | 1.08 | 0.97 | | | | | | | | | |
| 79 | Lys | 1.14 | 0.93 | | Arg | | | | | | | |
| 80 | Ile | 0.83 | 0.67 | | | | | | | | | |
| 81 | Ile | 0.05 | 0.08 | | | | | | | | | |
| 82 | Ser | 0.49 | 0.30 | | Thr | | | | | | | |
| 83 | Phe | 0.06 | 0.17 | | | | | | | | | |
| 84 | Lys | 0.51 | 0.42 | | | | | | | | | |
| 85 | Glu | 1.17 | 0.93 | | Asp | | | | | | | |
| 86 | Met | 0.45 | 0.41 | | Val, Asn | | | | | | | |
| 87 | Asn | 0.77 | 0.54 | | Gln | | | | | | | |
| 88 | Pro | 0.78 | 0.72 | | Ser | | | | | | | |
| 89 | Pro | 0.72 | 0.73 | | | | | | | | | |
| 90 | Asp | 0.93 | 0.66 | | | | | | | | | |
| 91 | Asn | 0.44 | 0.31 | | | | | | | | | |
| 92 | Ile | 0.59 | 0.49 | | Val | | | | | | | |
| 93 | Lys | 0.67 | 0.53 | | | | | | | | | |
| 94 | Asp | 0.83 | 0.56 | X | | | | | | | Lys | |
| 95 | Thr | 0.83 | 0.63 | X | | | | | | | Phe | X |
| 96 | Lys | 0.11 | 0.23 | | | | | | | | | |
| 97 | Ser | 0.77 | 0.62 | | | | | | | | | |
| 98 | Asp | 0.73 | 0.51 | | Glu, Asn | | | | | | | |
| 99 | Ile | 0.48 | 0.37 | | | | | | | | | |
| 100 | Ile | 0.20 | 0.15 | | | | | | | | | |
| 101 | Phe | 0.04 | 0.03 | | Tyr | | | | | X | | |
| 102 | Phe | 0.01 | 0.01 | | | | | | | | | |
| 103 | Gln | 0.19 | 0.14 | | | | | | | | | |
| 104 | Arg | 0.33 | 0.26 | X | | | | | | | Leu | |
| 105 | Ser | 0.16 | 0.13 | | | | | | | | | |
| 106 | Val | 0.29 | 0.31 | | | | | | | | | |
| 107 | Pro | 0.48 | 0.43 | | | | | | | | | |
| 108 | Gly | — | 0.83 | X | | | | | | | Ile | X |
| 109 | His | 1.11 | 1.04 | X | | | | | | | | X |
| 110 | Asp | 1.02 | 0.85 | X | Arg | | | | | | | X |
| 111 | Asn | 0.76 | 0.53 | X | | | | | | | Lys | |
| 112 | Lys | 0.53 | 0.39 | | | | | | | | | |
| 113 | Met | 0.06 | 0.04 | | | | | | | | | |
| 114 | Gln | 0.26 | 0.19 | | Asn | | | | | | | |
| 115 | Phe | 0.07 | 0.05 | | | | | | | | | |
| 116 | Glu | 0.13 | 0.09 | | | | | | | | | |
| 117 | Ser | 0.02 | 0.01 | | | | | | | | | |
| 118 | Ser | 0.42 | 0.33 | | Thr | | | | | | | |
| 119 | Ser | 0.16 | 0.30 | | | | | | | | | |
| 120 | Tyr | 0.50 | 0.42 | | Phe | | | | | | | |
| 121 | Glu | 0.82 | 0.73 | | Asp | | | | | | | |
| 122 | Gly | — | 0.48 | | | | | | | | | |
| 123 | Tyr | 0.57 | 0.46 | | Phe | | | | | | | |
| 124 | Phe | 0.36 | 0.29 | | Tyr | | | | | | | |
| 125 | Leu | 0.06 | 0.04 | | | | | | | | | |
| 126 | Ala | 0.12 | 0.07 | | | | | | | | | |
| 127 | Cys | 0.09 | 0.07 | | | | | | | | | |
| 128 | Glu | 0.55 | 0.41 | | | | | | | | | |
| 129 | Lys | 0.86 | 0.65 | X | | | | | | | Phe | |
| 130 | Glu | 1.00 | 0.74 | X | | | | | | | | X |
| 131 | Arg | 0.46 | 0.51 | X | | | | | | | Asp | |
| 132 | Asp | 0.57 | 0.52 | X | | | | | | | Leu | |
| 133 | Leu | 0.73 | 0.56 | X | | | | | | | Glu | |
| 134 | Phe | 0.49 | 0.42 | X | Tyr | | | | | | Ala | |
| 135 | Lys | 0.15 | 0.14 | | | | | | | | | |
| 136 | Leu | 0.16 | 0.14 | | | | | | | | | |
| 137 | Ile | 0.12 | 0.15 | | | | | | | | | |
| 138 | Leu | 0.52 | 0.40 | | | | | | | | | |
| 139 | Lys | 0.58 | 0.51 | | | | | | | | | |
| 140 | Lys | 1.03 | 0.93 | | | | | | | | | |
| 141 | Glu | 1.35 | 1.17 | | Lys, Asp | | | | | | | |
| 142 | Asp | 0.78 | 0.61 | | | | | | | | | |
| 143 | Glu | 1.32 | 1.12 | | | | | | | | | |
| 144 | Leu | 0.39 | 0.39 | | | | | | | | | |
| 145 | Gly | — | 0.10 | | | | | | | | | |
| 146 | Asp | 0.51 | 0.34 | | | | | | | | | |
| 147 | Arg | 0.05 | 0.04 | | | | | | | | | |
| 148 | Ser | 0.16 | 0.10 | X | Phe | X | | | | | | |
| 149 | Ile | 0.15 | 0.17 | | | | | | | | | |
| 150 | Met | 0.68 | 0.51 | X | | | | | | | Thr | |
| 151 | Phe | 0.38 | 0.41 | X | | | | | | | Ser | |
| 152 | Thr | 0.32 | 0.24 | | | | | | | | | |
| 153 | Val | 0.99 | 0.78 | | | | | | | | | |
| 154 | Gln | 0.93 | 0.87 | | Asn | | | | | | | |
| 155 | Asn | 0.96 | 0.76 | | Glu, Ser | | | | | | | |
| 156 | Glu | 0.61 | 0.68 | | Asp, Gln | | | | | | | |
| 157 | Asp | 1.36 | 1.51 | | Glu, Asn | | | | | | | |

The model predicts that several changes could be made in IL-18. Changes in non-surface exposed residues that could be made that would result in the high probability of retention of IL-18 activity with no changes in immunogenicity are:

Thr¹⁰ for Ser¹⁰

Val¹² for Ile¹²

Ser⁴⁵ for Thr⁴⁵

Tyr⁴⁷ for Phe⁴⁷

Phe⁵² for Tyr⁵²

Val⁶⁴ for Ile⁶⁴

And/or Tyr¹⁰¹ for Phe¹⁰¹

For SEQ ID NO: 2.

These compounds would be useful as IL-18 agonists, for raising anti-IL-18 antibodies, for assays for IL-18 or IL-18 binding proteins and for preparation of affinity columns for the purification of IL-18 binding proteins.

Changes in amino acids with a low percentage of surface exposure that could be made that would result in the high probability of retention of IL-18 activity with possible changes in immunogenicity are:

Val⁵ for Leu⁵

Val²⁰ for Leu²⁰

Ile²⁰ for Leu²⁰

Tyr²¹ for Phe²¹

Val²² for Ile²²

Ile⁶⁶ for Val⁶⁶

Thr⁷² for Ser⁷²

Phe¹⁴⁸ for Ser¹⁴⁸

These compounds would be useful as IL-18 agonists, for raising anti-IL-18 antibodies, for assays for I-18 or IL-18 binding proteins and for preparation of affinity columns for the purification of IL-18 binding proteins.

Changes that could be made in amino acids involved in receptor contact that would result in alteration of IL-18 activity by either increasing or decreasing binding of the IL-18 analog to the IL-18 receptor are:

Glu⁴ for Lys⁴

Ile⁶ for Glu⁶

Asp⁸ for Lys⁸

Ile¹³ for Arg¹³

Arg¹⁵ for Leu¹⁵

Lys¹⁷ for Asp¹⁷

Lys²⁷ for Arg²⁷

Ala³⁰ for Phe³⁰

Lys³⁵ for Asp³⁵

Phe³⁷ for Asp³⁷

Glu³⁸ for Cys³⁸

Ala³⁹ for Arg³⁹

Trp⁴⁰ for Asp⁴⁰

Glu⁵¹ for Met⁵¹

Gly⁵³ for Lys⁵³

Ile⁵⁶ for Gln⁵⁶

Ala⁵⁸ for Arg⁵⁸

Lys⁶² for Val⁶²

Lys⁹⁴ for Asp⁹⁴

Phe⁹⁵ for Thr⁹⁵

Leu¹⁰⁴ for Arg¹⁰⁴

Ile¹⁰⁸ for Gly¹⁰⁸

Lys¹¹¹ for Asn¹¹¹

Phe¹²⁹ for Lys¹²⁹

Asp¹³¹ for Arg¹³¹

Leu¹³² for Asp¹³²

Glu¹³³ for Leu¹³³

Ala¹³⁴ for Phe¹³⁴

Thr¹⁵⁰ for Met¹⁵⁰

Ser¹⁵¹ for Phe¹⁵¹

Depending on the alteration of receptor binding or receptor activity, these compounds would be useful as IL-18 agonists or antagonists, for preparation of antibodies against IL-18, in assays for IL-18 or IL-18 binding proteins and the preparation of affinity columns for the purification of IL-18 binding proteins.

Advantages:

The model described herein has as the advantage of allowing for predicting the effect of changing amino acids in IL-18 and allowing for the rationale design of new and potentially useful IL-18 muteins that do not exist in nature.

It will be clear that the invention can be practiced otherwise than as particularly described in the foregoing description and examples.

Numerous modifications and variations of the present invention are possible in light of the above teachings and, therefore, are within the scope of the appended claims.

What is claimed is:

1. At least one MUT-IL-18 nucleic acid, comprising or complementary to at least one polynucleotide encoding the amino acid sequence of SEQ ID NO: 1, wherein said amino acid sequence further comprises at least one mutation corresponding to at least one substitution selected from the group consisting of: Thr10 for Ser10; Val12 for Ile12; Ser45 for Thr45; Tyr47 for Phe47; Phe52 for Tyr52; Val64 for Ile64; Tyr101 for Phe101; Val5 for Leu5; Val20 for Leu20; Ile20 for Leu20; Tyr21 for Phe21; Val22 for Ile22; Ile66 for Val66; Thr72 for Ser72; Phe148 for Ser148, of SEQ ID NO: 1.

2. At least one MUT-IL-18 nucleic acid, comprising at least one polynucleotide encoding at least one MUT-IL-18 polypeptide, comprising at least 15 contiguous amino acids of SEQ ID NO: 1, wherein said amino acid sequence further comprises at least one mutation corresponding to at least one substitution selected from the group consisting of: Thr10 for Ser10; Val12 for Ile12; Ser45 for Thr45; Tyr47 for Phe47; Phe52 for Tyr52; Val64 for Ile64; Tyr101 for Phe101; Val5 for Leu5; Val20 for Leu20; Ile20 for Leu20;
Tyr21 for Phe21; Val22 for Ile22; Ile66 for Val66; Thr72 for Ser72; Phe148 for Ser148, of SEQ ID NO: 1.

3. At least one MUT-IL-18 nucleic acid, comprising at least one polynucleotide encoding at least one MUT-IL-18 polypeptide, comprising at least one extracellular, transmembrane or cytoplasmic domain of SEQ ID NO: 1, wherein said amino acid sequence further comprises at least one mutation corresponding to at least one substitution selected from the group consisting of: Thr10 for Ser10; Val12 for Ile12; Ser45 for Thr45; Tyr47 for Phe47; Phe52 for Tyr52; Val64 for Ile64; Tyr101 for Phe101; Val5 for Leu5; Val20 for Leu20; Ile20 for Leu20; Tyr21 for Phe21; Val22 for Ile22; Ile66 for Val66; Thr72 for Ser72; Phe148 for Ser148, of SEQ ID NO: 1.

4. At least one MUT-IL-18 nucleic acid, comprising at least one polynucleotide encoding at least one MUT-IL-18 polypeptide, comprising at least one polypeptide having at least 90-99% identity to an amino acid sequence comprising all of the contiguous amino acids of SEQ ID NO: 1, wherein said amino acid sequence further comprises at least one mutation corresponding to at least one substitution selected from the group consisting of: Thr10 for Ser10; Val12 for Ile12; Ser45 for Thr45; Tyr47 for Phe47; Phe52 for Tyr52; Val64 for Ile64; Tyr101 for Phe101; Val5 for Leu5; Val20 for Leu20; Ile20 for Leu20; Tyr21 for Phe21; Val22 for Ile22; Ile66 for Val66; Thr72 for Ser72; Phe148 for Ser148, of SEQ ID NO: 1.

5. At least one MUT-IL-18 polypeptide, comprising all of the contiguous amino acids of SEQ ID NO: 1, wherein said amino acid sequence further comprises at least one mutation corresponding to at least one substitution selected from the group consisting of: Thr10 for Ser10; Val12 for Ile12; Ser45 for Thr45; Tyr47 for Phe47; Phe52 for Tyr52; Val64 for Ile64; Tyr101 for Phe101; Val5 for Leu5; Val20 for Leu20; Ile20 for Leu20; Tyr21 for Phe21; Val22 for Ile22; Ile66 for Val66; Thr72 for Ser72; Phe148 for Ser148, of SEQ ID NO: 1.

6. At least one MUT-IL-18 polypeptide, comprising at least 15 contiguous amino acids of SEQ ID NO: 1, wherein said amino acid sequence further comprises at least one mutation corresponding to at least one substitution selected from the group consisting of: Thr10 for Ser10; Val12 for Ile12; Ser45 for Thr45; Tyr47 for Phe47; Phe52 for Tyr52; Val64 for Ile64; Tyr101 for Phe101; Val5 for Leu5; Val20 for Leu20; Ile20 for Leu20; Tyr21 for Phe21; Val22 for Ile22; Ile66 for Val66; Thr72 for Ser72; Phe148 for Ser148, of SEQ ID NO: 1.

7. At least one MUT-IL-18 polypeptide, comprising at least one extracellular, transmembrane or cytoplasmic domain of SEQ ID NO: 1, wherein said amino acid sequence further comprises at least one mutation corresponding to at least one substitution selected from the group consisting of: Thr10 for Ser10; Val12 for Ile12; Ser45 for Thr45; Tyr47 for Phe47; Phe52 for Tyr52; Val64 for Ile64; Tyr101 for Phe101; Val5 for Leu5for Leu20; Ile20 for Leu20; Tyr21 for Phe21; Val22 for Ile22; Ile66 for Val66;Thr72 ; Val20for Leu20; Ile20; for Leu20; Tyr21 for Phe21; Val22 for Ile22; Ile66 for Val66; Thr72 for Ser72; Phe148 for Ser148, of SEQ ID NO: 1.

8. At least one MUT-IL-18 polypeptide, comprising at least one polypeptide having at least 90-99% identity to an amino acid sequence comprising all of the contiguous amino acids of SEQ ID NO: 1, wherein said amino acid sequence further comprises at least one mutation corresponding to at least one substitution selected from the group consisting of: Thr10 for Ser10; Val12 for Ile12; Ser45 for Thr45; Tyr47 for Phe47; Phe52 for Tyr52; Val64 for Ile64; Tyr101 for Phe101; Val5 for Leu5; Val20 for Leu20; Ile20 for Leu20; Tyr21 for Phe21; Val22 for Ile22; Ile66 for Val66; Thr72 for Ser72; Phe148 for Ser148, of SEQ ID NO: 1.

9. A(n) MUT-IL-18 nucleic acid or MUT-IL-18 polypeptide according to any of claims 1-8, wherein said polypeptide has at least one activity of at least one MUT-IL-18 polypeptide.

10. A MUT-IL-18 antibody, comprising a monoclonal or polyclonal antibody, fusion protein, or fragment thereof, that specifically binds at least one MUT-IL-18 polypeptide according to any of claims 1-8.

11. A MUT-IL-18 nucleic acid encoding at least one MUT-IL-18 polypeptide or MUT-IL-18 antibody according to any of claim 1-10.

12. A MUT-IL-18 vector comprising at least one isolated nucleic acid according to any of claims 1-4 or encoding, or complementary to such nucleic acid encoding, a MUT-IL-18 according to any of claims 4-8.

13. A MUT-IL-18 host cell comprising an isolated nucleic acid according to claim 12.

14. A MUT-IL-18 host cell according to claim 13, wherein said host cell is at least one selected from COS-1, COS-7, HEK293, BHK21, CHO, BSC-1, Hep G2, 653, SP2/0, 293, NSO, DG44 CHO, CHO K1, HeLa, myeloma, or lymphoma cells, or any derivative, immortalized or transformed cell thereof.

15. A method for producing at least one MUT-IL-18 polypeptide or MUT-IL-18 antibody, comprising translating a nucleic acid according to claim 11 under conditions in vitro, in vivo or in situ, such that the MUT-IL-18 polypeptide is expressed in detectable or recoverable amounts.

16. A composition comprising at least one MUT-IL-18 nucleic acid, MUT-IL-18 polypeptide, or MUT-IL-18 antibody according to any of claims 1-10.

17. A composition according to claim 16, wherein said composition further comprises at least one pharmaceutically acceptable carrier or diluent.

18. A composition according to claim 16, further comprising at least one composition comprising an therapeutically effective amount of at least one compound, composition or polypeptide selected from at least one of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an opthalmic, otic or nasal drug, a topical drug, a nutritional drug, a cytokine, or a cytokine antagonist.

19. A composition according to claim 16, in a form of at least one selected from a liquid, gas, or dry, solution, mixture, suspension, emulsion or colloid, a lyophilized preparation, a powder.

20. A method for diagnosing or treating a MUT-IL-18 related condition in a cell, tissue, organ or animal, comprising
(a) contacting or administering a composition comprising an effective amount of at least one MUT-IL-18 nucleic acid, polypeptide or antibody according to any of claims 1-10, with, or to, said cell, tissue, organ or animal.

21. A method according to claim 20, wherein said effective amount is 0.001-50 mg of MUT-IL-18 antibody; 0.000001-500 mg of said MUT-IL-18; or 0 0001-100 μg of said MUT-IL-18 nucleic acid per kilogram of said cells, tissue, organ or animal.

22. A method according to claim 20, wherein said contacting or said administrating is by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

23. A method according to claim 20, further comprising administering, prior, concurrently or after said (a) contacting or administering, at least one composition comprising an effective amount of at least one compound or polypeptide selected from at least one of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an opthalmic, otic or nasal drug, a topical drug, a nutritional drug, a cytokine, or a cytokine antagonist.

24. A device, comprising at least one isolated MUT-IL-18 polypeptide, antibody or nucleic acid according to any of claims 1-10, wherein said device is suitable for contacting or administerting said at least one of said MUT-IL-18 polypeptide, antibody or nucleic acid, by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

25. An article of manufacture for human pharmaceutical or diagnostic use, comprising packaging material and a container comprising at least one isolated MUT-IL-18 polypeptide, antibody or nucleic acid according to any of claims 1-10.

26. The article of manufacture of claim 25, wherein said container is a component of a parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal delivery device or system.

27. A method for producing at least one isolated MUT-IL-18 polypeptide, antibody or nucleic acid according to any of claims 1-10, comprising providing at least one host cell, transgenic animal, transgenic plant, plant cell capable of expressing in detectable or recoverable amounts said polypeptide, antibody or nucleic acid.

28. At least one MUT-IL-18 polypeptide, antibody or nucleic acid, produced by a method according to claim 27.

29. At least one MUT-IL-18R nucleic acid, comprising or complementary to at least one polynucleotide encoding the amino acid sequence of SEQ ID NO: 2, further comprising at least one mutation corresponding to at least one substitution selected from the group consisting of: Glu4 for Lys4; Ile6 for Glu6; Asp8 for Lys8; Ile13 for Arg13; Arg15 for Leu15; Lys17 for Asp17; Lys27 for Arg27; Ala30 for Phe30; Lys35 for Asp35; Phe37 for Asp37; Glu38 for Cys38; Ala39 for Arg39; Trp40 for Asp40; Glu51 for Met51; Gly53 for Lys53; Ile56 for Gln56; Ala58 for Arg58; Lys62 for Val62; Lys94 for Asp94; Phe95 for Thr95; Leu104 for Arg104; Ile108 for Gly108; Lys111 for Asn111; Phe129 for Lys129; Asp131 for Arg131; Leu132 for Asp132; Glu133 for Leu133; Ala134 for Phe134; Thr150 for Met150; Ser151 for Phe151, of at least one of SEQ ID NO: 2.

30. At least one MUT-IL-18R nucleic acid, comprising at least one polynucleotide encoding at least one MUT-IL-18R polypeptide, comprising at least 15 contiguous amino acids of SEQ ID NO: 2, further comprising at least one mutation corresponding to at least one substitution selected from the group consisting of: Glu4 for Lys4; Ile6 for Glu6; Asp8 for Lys8; Ile13 for Arg13; Arg15 for Leu15; Lys17 for Asp17; Lys27 for Arg27; Ala30 for Phe30; Lys35 for Asp35; Phe37 for Asp37; Glu38 for Cys38; Ala39 for Arg39; Trp40 for Asp40; Glu51 for Met51; Gly53 for Lys53; Ile56 for Gln56; Ala58 for Arg58; Lys62 for Val62; Lys94 for Asp94; Phe95 for Thr95; Leu104 for Arg104; Ile108 for Gly108; Lys111 for Asn111; Phe129 for Lys129; Asp131 for Arg131; Leu132 for Asp132; Glu133 for Leu133; Ala134 for Phe134; Thr150 for Met150; Ser151 for Phe151, of at least one of SEQ ID NO: 2.

31. At least one MUT-IL-18R nucleic acid, comprising at least one polynucleotide encoding at least one MUT-IL-18R polypeptide, comprising at least one extracellular, transmembrane or cytoplasmic domain of SEQ ID NO: 2, further comprising at least one mutation corresponding to at least one substitution selected from the group consisting of: Glu4 for Lys4; Ile6 for Glu6; Asp8 for Lys8; Ile13 for Arg13; Arg15 for Leu15; Lys17 for Asp17; Lys27 for Arg27; Ala30 for Phe30; Lys35 for Asp35; Phe37 for Asp37; Glu38 for Cys38; Ala39 for Arg39; Trp40 for Asp40; Glu51 for Met51; Gly53 for Lys53; Ile56 for Gln56; Ala58 for Arg58; Lys62 for Val62; Lys94 for Asp94; Phe95 for Thr95; Leu104 for Arg104; Ile108 for Gly108; Lys111 for Asn111; Phe129 for Lys129; Asp131 for Arg131; Leu132 for Asp132; Glu133 for Leu133; Ala134 for Phe134; Thr150 for Met150; Ser151 for Phe151, of at least one of SEQ ID NO: 2.

32. At least one MUT-IL-18R nucleic acid, comprising at least one polynucleotide encoding at least one MUT-IL-18R polypeptide, comprising at least one polypeptide having at least 90-99% identity to an amino acid sequence comprising all of the contiguous amino acids of SEQ ID NO: 2, further comprising at least one mutation corresponding to at least one substitution selected from the group consisting of: Glu4 for Lys4; Ile6 for Glu6; Asp8 for Lys8; Ile13 for Arg13; Arg15 for Leu15; Lys17 for Asp17; Lys27 for Arg27; Ala30 for Phe30; Lys35 for Asp35; Phe37 for Asp37; Glu38 for Cys38; Ala39 for Arg39; Trp40 for Asp40; Glu51 for Met51; Gly53 for Lys53; Ile56 for Gln56; Ala58 for Arg58; Lys62 for Val62; Lys94 for Asp94; Phe95 for Thr95; Leu104 for Arg104; Ile108 for Gly108; Lys111 for Asn111; Phe129 for Lys129; Asp131 for Arg131; Leu132 for Asp132; Glu133 for Leu133; Ala134 for Phe134; Thr150 for Met150; Ser151 for Phe151, of at least one of SEQ ID NO: 2.

33. At least one MUT-IL-18R polypeptide, comprising all of the contiguous amino acids of SEQ ID NO: 2, further comprising at least one mutation corresponding to at least one substitution selected from the group consisting of: Glu4 for Lys4; Ile6 for Glu6; Asp8 for Lys8; Ile13 for Arg13; Arg15 for Leu15; Lys17 for Asp17; Lys27 for Arg27; Ala30 for Phe30; Lys35 for Asp35; Phe37 for Asp37; Glu38 for Cys38; Ala39 for Arg39; Trp40 for Asp40; Glu51 for Met51; Gly53 for Lys53; Ile56 for Gln56; Ala58 for Arg58; Lys62 for Val62; Lys94 for Asp94; Phe95 for Thr95; Leu104 for Arg104; Ile108 for Gly108; Lys111 for Asn111; Phe129 for Lys129; Asp131 for Arg131; Leu132 for Asp132; Glu133 for Leu133; Ala134 for Phe134; Thr150 for Met150; Ser151 for Phe151, of at least one of SEQ ID NO: 2.

34. At least one MUT-IL-18R polypeptide, comprising at least 15 contiguous amino acids of SEQ ID NO: 2, further comprising at least one mutation corresponding to at least one substitution selected from the group consisting of: Glu4 for Lys4; Ile6 for Glu6; Asp8 for Lys8; Ile13 for Arg13; Arg15 for Leu15; Lys17 for Asp17; Lys27 for Arg27; Ala30 for Phe30; Lys35 for Asp35; Phe37 for Asp37; Glu38 for Cys38; Ala39 for Arg39; Trp40 for Asp40; Glu51 for Met51; Gly53 for Lys53; Ile56 for Gln56; Ala58 for Arg58; Lys62 for Val62; Lys94 for Asp94; Phe95 for Thr95; Leu104 for Arg104; Ile108 for Gly108; Lys111 for Asn111; Phe129 for Lys129; Asp131 for Arg131; Leu132 for Asp132; Glu133 for Leu133; Ala134 for Phe134; Thr150 for Met150; Ser151 for Phe151, of at least on of SEQ ID NO: 2.

35. At least one MUT-IL-18R polypeptide, comprising at least one extracellular, transmembrane or cytoplasmic domain of SEQ ID NO: 2, further comprising at least one mutation corresponding to at least one substitution selected from the group consisting of: Glu4 for Lys4; Ile6 for Glu6; Asp8 for Lys8; Ile13 for Arg13; Arg15 for Leu15; Lys17 for Asp17; Lys27 for Arg27; Ala30 for Phe30; Lys35 for Asp35; Phe37 for Asp37; Glu38 for Cys38; Ala39 for Arg39; Trp40 for Asp40; Glu51 for Met51; Gly53 for Lys53; Ile56 for Gln56; Ala58 for Arg58; Lys62 for Val62; Lys94 for Asp94; Phe95 for Thr95; Leu104 for Arg104; Ile108 for Gly108; Lys111 for Asn111; Phe129 for Lys129; Asp131 for Arg131; Leu132 for Asp132; Glu133 for Leu133; Ala134 for Phe134; Thr150 for Met150; Ser151 for Phe151, of at least one of SEQ ID NO: 2.

36. At least one MUT-IL-18R polypeptide, comprising at least one polypeptide having at least 90-99% identity to an amino acid sequence comprising all of the contiguous amino acids of SEQ ID NO: 2, further comprising at least one mutation corresponding to at least one substitution selected from the group consisting of: Glu4 for Lys4; Ile6 for Glu6; Asp8 for Lys8; Ile13 for Arg13; Arg15 for Leu15; Lys17 for Asp17; Lys27 for Arg27; Ala30 for Phe30; Lys35 for Asp35; Phe37 for Asp37; Glu38 for Cys38; Ala39 for Arg39; Trp40 for Asp40; Glu51 for Met51; Gly53 for Lys53; Ile56 for Gln56; Ala58 for Arg58; Lys62 for Val62; Lys94 for Asp94; Phe95 for Thr95; Leu104 for Arg104; Ile108 for Gly108; Lys111 for Asn111; Phe129 for Lys129; Asp131 for Arg131; Leu132 for Asp132; Glu133 for Leu133; Ala134 for Phe134; Thr150 for Met150; Ser151 for Phe151, of at least one of SEQ ID NO: 2.

37. A(n) MUT-IL-18R nucleic acid or MUT-IL-18R polypeptide according to any of claims 1-8, wherein said polypeptide has at least one activity of at least one MUT-IL-18R polypeptide.

38. A MUT-IL-18R antibody, comprising a monoclonal or polyclonal antibody, fusion protein, or fragment thereof, that specifically binds at least one MUT-IL-18R polypeptide according to any of claims 1-8.

39. A MUT-IL-18R nucleic acid encoding at least one MUT-IL-18R polypeptide or MUT-IL-18R antibody according to any of claim 1-10.

40. A MUT-IL-18R vector comprising at least one isolated nucleic acid according to any of claims 1-4 or encoding, or complementary to such nucleic acid encoding, a MUT-IL-18R according to any of claims 4-8.

41. A MUT-IL-18R host cell comprising an isolated nucleic acid according to claim 12.

42. A MUT-IL-18R host cell according to claim 13, wherein said host cell is at least one selected from COS-1, COS-7, HEK293, BHK21, CHO, BSC-1, Hep G2, 653, SP2/0, 293, NSO, DG44 CHO, CHO K1, HeLa, myeloma, or lymphoma cells, or any derivative, immortalized or transformed cell thereof.

43. A method for producing at least one MUT-IL-18R polypeptide or MUT-IL-8R antibody, comprising translating a nucleic acid according to claim 11 under conditions in vitro, in vivo or in situ, such that the MUT-IL-18R polypeptide is expressed in detectable or recoverable amounts.

44. A composition comprising at least one MUT-IL-18R nucleic acid, MUT-IL-18R polypeptide, or MUT-IL-18R antibody according to any of claims 1-10.

45. A composition according to claim 16, wherein said composition further comprises at least one pharmaceutically acceptable carrier or diluent.

46. A composition according to claim 16, further comprising at least one composition comprising an therapeutically effective amount of at least one compound, composition or polypeptide selected from at least one of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an opthalmic, otic or nasal drug, a topical drug, a nutritional drug, a cytokine, or a cytokine antagonist.

47. A composition according to claim 16, in a form of at least one selected from a liquid, gas, or dry, solution, mixture, suspension, emulsion or colloid, a lyophilized preparation, a powder.

48. A method for diagnosing or treating a MUT-IL-18R related condition in a cell, tissue, organ or animal, comprising (a) contacting or administering a composition comprising an effective amount of at least one MUT-IL-18R nucleic acid, polypeptide or antibody according to any of claims 1-10, with, or to, said cell, tissue, organ or animal.

49. A method according to claim 20, wherein said effective amount is 0.001-50 mg of MUT-IL-18R antibody; 0.000001-500 mg of said MUT-IL-18R; or 0.0001-100 μg of said MUT-L-18R nucleic acid per kilogram of said cells, tissue, organ or animal.

50. A method according to claim 20, wherein said contacting or said administrating is by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

51. A method according to claim 20, further comprising administering, prior, concurrently or after said (a) contacting or administering, at least one composition comprising an effective amount of at least one compound or polypeptide selected from at least one of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an opthalmic, otic or nasal drug, a topical drug, a nutritional drug, a cytokine, or a cytokine antagonist.

52. A device, comprising at least one isolated MUT-IL-18R polypeptide, antibody or nucleic acid according to any of claims 1-10, wherein said device is suitable for contacting or administerting said at least one of said MUT-IL-18R polypeptide, antibody or nucleic acid, by at least one mode selected from parenteral, subcutaneous, intramuscular, intravenous, intrarticular, intrabronchial, intraabdominal, intracapsular, intracartilaginous, intracavitary, intracelial, intracelebellar intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

53. An article of manufacture for human pharmaceutical or diagnostic use, comprising packaging material and a container comprising at least one isolated MUT-IL-18R polypeptide, antibody or nucleic acid according to any of claims 1-10.

54. The article of manufacture of claim 25, wherein said container is a component of a parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal delivery device or system.

55. A method for producing at least one isolated MUT-IL-18R polypeptide, antibody or nucleic acid according to any of claims 1-10, comprising providing at least one host cell, transgenic animal, transgenic plant, plant cell capable of expressing in detectable or recoverable amounts said polypeptide, antibody or nucleic acid.

56. At least one MUT-IL-18R polypeptide, antibody or nucleic acid, produced by a method according to claim 27.

57. Any invention described herein.


SEQUENCE LISTING (2 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 224 aa, Homo sapiens
ESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNE